(19) United States
(12) Patent Application Publication
(10) Pub. No.: US 2014/0377288 A1
(43) Pub. Date: Dec. 25, 2014

(54) COMPOSITIONS AND METHODS RELATED TO DNA DAMAGE REPAIR

(71) Applicants: Fernanda I. Staquicini, Albuquerque, NM (US); Renata Pasqualini, Los Ranchos, NM (US); Wadih Arap, Los Ranchos, NM (US)

(72) Inventors: Fernanda I. Staquicini, Albuquerque, NM (US); Renata Pasqualini, Los Ranchos, NM (US); Wadih Arap, Los Ranchos, NM (US)

(73) Assignee: THE BOARD OF REGENTS OF THE UNIVERSITY OF TEXAS SYSTEM, Austin, TX (US)

(21) Appl. No.: 14/343,943

(22) PCT Filed: Sep. 14, 2012
(86) PCT No.: PCT/US12/55598

§ 371 Date: Aug. 1, 2014

Related U.S. Application Data
(60) Provisional application No. 61/535,811, filed on Sep. 16, 2011.

(51) Int. Cl. C07K 16/28

(52) U.S. Cl. CPC C07K 16/28; C07K 2317/56; C07K 2317/76; A61K 2039/505
USPC 424/178.1; 530/387.9; 435/69.6

(57) ABSTRACT

The present invention provides compositions and methods for treating a cancer associated with elevated expression and/or activity of receptor tyrosine kinases (e.g., Eph receptors), such as EphA5. In some embodiments, the present invention provides compositions and methods for identifying elevated expression or activity of receptor tyrosine kinases (e.g., Eph receptors), such as EphA5.

The application claims priority to U.S. Provisional Patent Application No. 61/535,811 filed Sep. 16, 2011, which is incorporated herein by reference in its entirety.

### GOVERNMENT SUPPORT

The invention was made with government support under Grant No. P50 CA70907 and P30 CA016672 awarded by the National Cancer Institute and the National Institutes of Health and under Grant No. W81XWH-05-2-0027 awarded by the Department of Defense. The government has certain rights in the invention.

### BACKGROUND

Cells are continually exposed to factors, such as intracellular reactive species and environmental agents, which are capable of causing DNA damage. Typically, the potentially mutagenic consequences of DNA damage are minimized by DNA repair pathways, which are broadly characterized into three forms: base excision repair (BER), mismatch repair (MMR), and nucleotide excision repair (NER) (see, e.g., Wood et al., Science, 291: 1284-1289 (2001)). Deficiencies in DNA damage repair can contribute to a variety of diseases, including the development and/or pathogenesis of cancer as well as many genetic disorders.

The hallmark of cancer is continuous cell division, which requires both the replication of the cell's DNA as well as the transcription and translation of many genes needed for continued growth of the cell. It is contemplated that a chemical that damages DNA would have the potential to inhibit the spread of cancer. Indeed, it has been demonstrated that several chemotherapeutic DNA damaging agents are useful for inhibiting the spread of cancerous cells, such as, for example, purine analogs, pyrimidine analogs, alkylating agents, crosslinking agents, topoisomerases, among others. Radiotherapy also functions by overwhelming the capacity of the cell to repair DNA damage, resulting in cell death. However, both chemotherapeutic agents and radiotherapies generally target any rapidly proliferating cell, whether it is cancerous or not, including intestinal cells, bone marrow cells, and hair follicles, leading to generalized and often serious side effects in patients. Furthermore, DNA damaging agents themselves have the potential to create additional cancers, such as leukemia in patients.

Many patients with cancer develop resistance to therapies, such as chemotherapy, and develop metastatic tumors, and ultimately die of their disease.

### SUMMARY

The present invention encompasses the recognition that receptor tyrosine kinases, such as Eph receptors (e.g., EphA5), may play a role in regulation of DNA damage repair. For example, the present inventors have surprisingly found that certain cancers, e.g., certain human lung cancers, overexpress the receptor tyrosine kinase EphA5, which facilitates DNA damage repair and promotes resistance of the cells to ionizing radiation and selected chemotherapeutic agents. Indeed, the present inventors have found that, in response to genotoxic stress, EphA5 is phosphorylated and present in the nucleus of cancer cells. The present disclosure further encompasses the finding that a direct interaction between EphA5 and activated ATM occurs at sites of DNA damage, which is contemplated to enhance DNA damage repair and thereby render the cells resistant both to ionizing radiation therapy and chemotherapeutics which induce DNA damage.

Among other things, the present invention provides compositions and methods for treating a cancer associated with elevated levels, activity and/or phosphorylation of Eph receptors, such as EphA5. In some embodiments, the present invention provides compositions and methods for identifying elevated levels, activity and/or phosphorylation of Eph receptors, such as EphA5.

In some embodiments, the present invention provides an isolated antibody, or an antigen-binding fragment thereof, characterized in that it binds to human EphA5 protein and characterized in that: (a) it decreases levels of phosphorylated EphA5 in the nucleus of a cell expressing EphA5 as compared to a control; (b) it decreases binding of EphA5 protein to pATM protein in a cell expressing EphA5 as compared to a control; (c) it competes with the 11C12 antibody or an EphA5 ligand for binding to EphA5; (d) it decreases cell growth, proliferation or cell survival in a cell expressing EphA5 as compared to a control; or (e) a combination of (a)-(d). In some embodiments, the present invention provides methods of producing such isolated antibodies, or antigen-binding fragments thereof by providing a cell expressing the isolated antibody; and culturing the cell under conditions permissive for expression of the antibody thereby producing the antibody (antigen-binding fragment thereof).

In some embodiments, a pharmaceutical composition is provided for treating a cell hyperproliferative disorder associated with elevated levels of EphA, such as EphA5. For example, such a pharmaceutical composition can comprise a therapeutically effective amount of an agent that reduces the expression of the EphA or reduces the activation of the EphA (e.g., reduces EphA phosphorylation or reduces the level of EphA in the nucleus of EphA expressing cells).

In some embodiments there is provided a method of treating a cell hyperproliferative disorder comprising administering a therapeutically effective amount of an agent that reduces the expression of the EphA or reduces the activation of the EphA (e.g., reduces EphA phosphorylation or reduces the level of EphA in the nucleus of EphA expressing cells). For example, such a method may be used in the treatment of a hyperproliferative disorder associated with elevated EphA expression or activation.

In some embodiments there is provided a method of treating patient having a cell hyperproliferative disorder wherein the cells associated with said disorder were previously determined to express elevated levels of EphA or have an elevated EphA activity (e.g., elevated levels of phosphorylated or nuclear EphA), the method comprising administering an agent that reduces the expression level or activity of EphA

Without wishing to be bound by any particular theories, it is contemplated that compositions that target receptor tyrosine kinases, such as EphA or in particular EphA5, may act synergistically with other DNA damage agents for cancer treatment. Thus, in a some embodiments the invention provides a method of treating a patient having a cell hyperproliferative disorder, comprising administering in combination: an agent that reduces expression or activity of EphA and a DNA damaging agent, such radiation therapy or DNA damaging chemotherapeutic. In certain aspects, provided methods include administering in combination an agent that reduces expression or activity of EphA5 and radiation therapy. In some aspects, provided methods include administering in combination an agent that reduces expression or activity of EphA5 and a chemotherapeutic agent.

Thus, in some embodiments, provided methods comprise administering in combination an agent that reduces the presence of phosphorylated EphA5 and radiation therapy. In some embodiments, provided methods include administering in combination an agent that reduces the presence of phosphorylated EphA5 and a chemotherapeutic agent.

In some embodiments, provided methods include administering an agent that reduces expression or activity of EphA5 in the absence of radiation therapy. In certain embodiments, provided methods include administering an agent that reduces expression or activity of EphA5 in the absence of a chemotherapeutic agent.

In some embodiments, provided methods include administering an agent that reduces the presence of phosphorylated EphA5 in the absence of radiation therapy. In certain embodiments, provided methods include administering an agent that reduces the presence of phosphorylated EphA5 in the absence of a chemotherapeutic agent.

In some embodiments there is provided a method for sensitizing a cell to DNA damaging agents or reversing resistance to DNA damaging agents comprising contacting the cell with an agent that reduces EphA expression or activity.

In some embodiments there is provided a method of identifying a patient likely to benefit from therapy (such as a radiation therapy or an EphA-targeted therapy), the method comprising steps of: obtaining an expression or activity level of EphA from a patient sample; and determining that the level of EphA expression or activity is elevated as compared to a control level. Similarly a method of identifying a patient likely to benefit from therapy can comprise obtaining an EphA sequence from a patient sample and determining a difference in sequence in the obtained EphA5 sequence as compared to a control sample.

Among other things, the present invention provides methods of identifying a patient population likely to benefit from therapy, comprising steps of obtaining an expression level, an activity level, and/or sequence of EphA5 in one or more samples obtained from a patient, and determining a difference in the expression level, activity level and/or sequence of EphA5 in at least one sample obtained from a patient as compared to a control sample. In some embodiments, the present invention provides methods of identifying a patient population likely to benefit from therapy comprising steps of obtaining a level of phosphorylation of EphA5 in one or more samples obtained from a patient, and determining that the detected level is elevated in at least one sample obtained from the patient as compared to that observed in a control sample.

Certain embodiments of the invention also provide methods of identifying agents that regulate EphA expression or activation. For example, such a method can comprise providing a collection of one or more test agents; contacting the one or more test agents with a system comprising EphA, or a portion thereof; and comparing the expression or activation of EphA (e.g., phosphorylation of EphA5) in the presence and absence of the one or more test agents. Similarly agents that regulate EphA5 expression or activation can be identified by providing a collection of one or more test agents; providing a system comprising EphA5, or a portion thereof, and an EphA5 ligand (such as ephrinA3) or the 11C12 antibody, wherein the EphA5 and EphA5 ligand interact with one another; contacting the one or more test agents with the system; and determining whether the one or more test agents competes the interaction of the EphA5 and the EphA5 ligand or the 11C12 antibody.

In some embodiments, the present invention provides methods of identifying agents that regulate phosphorylation of EphA5. For example, in some embodiments, methods of identifying agents that regulate phosphorylation of EphA5 include providing a collection of one or more test agents, contacting the one or more test agents with a system comprising EphA5, or a portion thereof, and comparing the phosphorylation of EphA5 in the presence and absence of the one or more test agents.

In some embodiments a method of detecting a cancer cell in a sample is provided comprising contacting a sample with an EphA5 binding agent characterized in that it competes with an EphA5 ligand or the 11C12 antibody for binding to EphA5 and determining level of binding of the EphA5 binding agent.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWING

The following drawings form part of the present specification and are included to further demonstrate certain aspects of the present invention. The invention may be better understood by reference to one or more of these drawings in combination with the detailed description of specific embodiments presented herein.

FIG. 1: Expression of EphA5 in wild-type H460 lung cancer cells, control cells infected with a lentivirus expressing a negative control shRNA, and cells infected with lentiviruses expressing two independent shRNA sequences targeting EphA5.

FIG. 2: Functional evaluation of EphA5 activity in human lung cancer. FIG. 2A, Cell cycle analysis in proliferating lung cancer cells (control shRNA, EphA5-shRNA 1 and EphA5-shRNA 2) was performed by BrdU incorporation and DNA content (upper panel). BrdU immunostaining served to calculate the percentage of cells in S phase (lower panel). FIG. 2B, Propidium iodide (PI) staining showing accumulation of cells in G0/G1 phase after partial cell cycle synchronization. FIG. 2C-E, Activation of IR-induced cell cycle checkpoints was evaluated after treatment of cells with 3 Gy (FIG. 2C), 5 Gy (FIG. 2D) and 10 Gy (FIG. 2E). Control cells arrested cycling in all experimental conditions, an indication of functional DNA-repair machinery. In contrast, EphA5-negative cells did not arrest cell cycle after 3 Gy (FIG. 2C) and 5 Gy (FIG. 2D) of irradiation, data indicative of impaired G1/S checkpoint. Alterations in G2/M checkpoint were not detected in control or EphA5-negative tumor cells (FIG. 2E). FIG. 2F, Increased radio sensitivity of EphA5-negative tumor cells by clonogenic assay. FIG. 2G, Ectopic expression of human EphA5 confers relative radio resistance to normal pulmonary fibroblasts.

FIG. 3: EphA5-negative human lung cancer cell lines are sensitive to ionizing radiation. To confirm the role of EphA5 in the cellular response to DNA damage, survival following IR was assayed of control and EphA5-negative H1299 (FIG. 3A), H522 (FIG. 3B) and A549 (FIG. 3C) human lung cancer cells using a clonogenic (colony formation) assay. Compared to their respective controls, H1299 and H522 EphA5-negative cells showed a marked reduction in survival upon IR-induced DNA damage, again supporting a role for EphA5 in DNA damage response. The effect of irradiation on the survival of A549 control cells was not different from that of A549 EphA5-negative cells.

FIG. 4: Control tumor cells and EphA5-negative cells were treated with hydroxyurea for 24 h followed by release into growth factor-containing media. Progression of cells through the cell cycle phases was evaluated by analysis of DNA content 3 h, 6 h, 8 h, and 24 h post-release. The percentage of cells in each phase of the cell cycle is shown.

FIG. 5: Direct molecular interaction between EphA5 and activated ATM in sites of DNA injury. FIG. 5A-F, Confocal analysis of EphA5 expression and distribution in lung cancer cells (H460). FIG. 5A, Differential contrast image (DIC) and confocal image of EphA5 in untreated tumor cells reveal accumulation of the receptor in the cytoplasmic and perinuclear regions. FIG. 5B, Z-projection of EphA5-expressing tumor cells shown in (FIG. 5A). White dashed lines point to orthogonal planes, confirming that EphA5 is distributed in the perinuclear region of the cytoplasm. White arrowheads point to nuclear EphA5. FIG. 5C, Co-immunostaining of EphA5 and pATM in untreated cancer cells. FIG. 5D-E, Co-immunostaining of EphA5 and pATM after 10 min of cell irradiation (3 Gy). White arrows point to nuclear foci of active DNA repair containing both pATM and EphA5. FIG. 5E, Orthogonal planes of the confocal image showed in (FIG. 5D) that confirm the co-localization of pATM and EphA5 at sites of DNA injury and repair. FIG. 5F, Co-localization of EphA5 and pATM in the nucleus of irradiated cancer cells 6 h after treatment with 3 Gy. Scale bar, 10 p.m. FIG. 5G-H, Quantification of p-ATM foci formation in control cells and EphA5-negative tumor cells treated with 3 Gy and evaluated after 10 min (FIG. 5G) and 6 h (FIG. 5H) of treatment. FIG. 5I-J, Direct physical interaction between pATM and the cytoplasmic domain of EphA5. Either pATM (FIG. 5I) or ATM (FIG. 5J) were immunocaptured from total cell extracts and subjected to binding to active domains of EphA5. Only the complete cytoplasmic domain of EphA5 interacts with pATM, in a concentration-dependent manner, suggesting specificity. FIG. 5K-L, Solution binding assays between ATM, pATM, and EphA5 domains confirm binding specificity of pATM and the cytoplasmic domain of EphA5. Arrow points to the cytoplasmic domain of EphA5 co-immunoprecipitated with pATM. FIG. 5L, Controls to demonstrate the presence of ATM and pATM in each experimental condition (left panels) and to rule out the possibility of non-specific interactions between pATM and the GST tag (right panel).

FIG. 6: Nuclear import and phosphorylation of EphA5. FIG. 6A, Cytoplasmic (C) and nuclear (N) proteins were isolated from non-irradiated cancer cells or from cancer cells irradiated with 3 Gy and were subjected to immunoprecipitation with an anti-EphA5 antibody. Detection of phosphorylated and total EphA5 was performed with an anti-pTyr antibody or an antibody against EphA5. Phosphorylated EphA5 was detected only in the nuclear fraction of the irradiated cancer cells. The cytoplasmic protein MEK1/2 and the nuclear protein Orc2 served as controls for purity (right panel). FIG. 6B-C, Surviving fractions of H460 (FIG. 6B) and H522 (FIG. 6C) lung cancer cell lines treated with the monoclonal antibody 11C12 and subjected to increasing doses of IR. Isotype IgG was used as a negative control. FIG. 6D, Representative clonogenic assays depicted in (FIG. 6B). FIG. 6E, The monoclonal antibody 11C12 inhibits phosphorylation of EphA5 upon treatment with IR. One hour before irradiation, cancer cells were treated with increasing doses of 11C12. Cytoplasmic (C) and nuclear (N) fractions were collected 30 min post irradiation. Non-irradiated cancer cells served as negative controls. Phosphorylated EphA5 was detected only in the nuclear fraction of non-treated tumor cells.

FIG. 7: FIG. 7A, Ligand-induced phosphorylation of EphA5. H460 lung cancer cells were treated for 20 min with 200 μg/mL of ephrin A3, and total cell extract was collected immediately after treatment (time point 0), or 5, 10 and 30 min after removal of the ligand. Extracted proteins were subjected to immunoprecipitation with an anti-EphA5 antibody, separated by electrophoresis, transferred to a nitrocellulose membrane and developed with anti-pTyr (for phosphorylated EphA5) or an antibody against EphA5. FIG. 7B, Nuclear import and phosphorylation of EphA5 in cells treated with the endogenous native ligand ephrin A3. FIG. 7C Treatment of H460 human lung cancer cells with control IgG does not inhibit phosphorylation of EphA5 upon ionizing radiation. Phosphorylated EphaA5 is indicated by the boxed areas.

FIG. 8: Evaluation of EphA5 expression in a comprehensive human lung cancer TMA. (A) Overall assessment of EphA5 expression in ACC and SCC samples. High levels of EphA5 expression were predominantly associated with SCC (80% of all cases analyzed) versus ACC (60% of all cases analyzed), * P<0.001; Wilcoxon rank sum test. (B) Representative microphotographs of EphA5 staining in ACC (top) and SCC (bottom) (magnification ×200). Insets: irrelevant IgG used as negative control. (C) Expression of EphA5 in patients receiving irradiation treatment prior to surgical resection of lung cancer. High levels of EphA5 expression are directly associated with radiotherapy failure, * P=0.0021; Log-rank Test. (D and E) Locoregional recurrence (D) and cumulative overall patient survival (E) rates as a function of EphA5 expression score.

FIG. 9: Depicts the results of an ELISA assay detecting 11C12 antibody binding in the presence of decreasing amounts of human EphA5 (circles) or murine EphA5 (squares). No binding of 11C12 to murine EphA5 was evident.

FIG. 10: Further 11C12 epitope mapping on human EphA5. ELISA assays were performed with decreasing amount of 11C12 antibody in the presence of various portions of human EphA5 fused to GST. GST fusion proteins included EphA5 sequence from the regions corresponding to amino acids 304-467 (SEQ ID NO:3) and are numbered relative to this region. GST fusion proteins included GST-hEphA5 (1-24 aa) (SEQ ID NO:4); GST-hEphA5 (1-48 aa) (SEQ ID NO:5); GST-hEphA5 (1-72 aa) (SEQ ID NO:6); GST-hEphA5 (1-95 aa) (SEQ ID NO:7); GST-hEphA5 (1-120 aa) (SEQ ID NO:8); GST-hEphA5 (1-144 aa) (SEQ ID NO:9); and GST-hEphA5 (1-164 aa) (SEQ ID NO:10). The greatest binding was observed with GST-hEphA5 (1-72 aa) indicating that the primary 11C12 binding eptiope is in the first 72 amino acids of the region 304-467 of the hEphA5 protein.

FIG. 11: The 11C12 monoclonal antibody reduces cancer cell adhesion and proliferation in culture. FIG. 11A, H460 cells were seeded in the presence of increasing concentrations of 11C12. The number of cell colonies formed were counted 10 days after the initial plating and plotted. FIG. 11B, H460 cells were grown in the presence of 100 μg/well of 11C12 or control IgG and cell proliferation was measured in real time by xCELLigence System assay (Roche). FIG. 11C, Cells were plated in the presence of 11C12 or a control IgG (each at 100 μg/well). Adhesion of the cells to the culture plate was assessed in real time during the first 6 hours of the experiment.

FIG. 12: Mechanism of EphA5-induced senescence in human lung cancer cells. Wild type p53 H460 cells (FIG. 12A), H1299 p53 null cells (FIG. 12B) and H522 (p53 mutated) (FIG. 12C) were used to demonstrate that EphA5-induced senescence depends on the presence and/or activity of p53. EphA5-positive and EphA5-negative cells were irradiated and stained for β-galactosidase activity at pH 6, a known characteristic of senescent cells not found in presenescent, quiescent or immortal cells. Approximately 60% of H460 (p53 wild-type) cells became senescent after treatment with IR. Both p53 null (H1299) and mutated (H522) cells did not become senescent and entered apoptosis.

FIG. 13: Nude mice were subcutaneously inoculated with human H460 lung cancer cells. Two weeks post inoculation the tumor bearing mice were administered 3 μg of fluorophore-labeled (IRDye®-labeled) 11C12 antibody or a labeled control IgG. Images of the mice were acquired on a Pearl imager 48- or 72-hours post antibody administration (left and right panels, respectively).

FIG. 14: Nude mice were inoculated with H460 cancer cells and tumor growth and animal survival was assessed for a period of 30 days. Groups of at least 8 mice were treated with 5 mg/kg of 11C12 or control antibody either alone or in conjunction with radiation. Radiation was applied 6 hours after antibody administration and was repeated at a dose of 3 Gy/day for three consecutive days (for a total 9 Gy radiation dose). FIG. 14A, Daily tumor volumes in the animals were plotted in each case. Data are reported as mean tumor volumes ±SD. FIG. 14B, Kaplan-Meier survival analysis. Combined treatment of 11C12 plus radiation therapy significantly improved the overall survival of tumor-bearing mice (Log-rank Test, *P=0.0061).

FIG. 15: Nude mice were inoculated with H460 cancer cells and tumor growth and animal survival was assessed for a period of 26 days. Groups of at least 8 mice were treated with 5 mg/kg of 7A5 or control antibody either alone or in conjunction with radiation. Radiation was applied 6 hours after antibody administration and was repeated at a dose of 3 Gy/day for three consecutive days (for a total 9 Gy radiation dose). FIG. 15A, Daily tumor volumes in the animals were plotted in each case. Data are reported as mean tumor volumes ±SD. FIG. 15B, Kaplan-Meier survival analysis. Combined treatment of 7A5 plus radiation therapy significantly improved the overall survival of tumor-bearing mice (Log-rank Test, *P=0.0096).

FIG. 16: Combined treatment of H460-derived human lung cancer xenografts with 11C12 and ionizing radiation (IR) reduces cell proliferation in vivo. (FIG. 16A) Gamma radiation was locally delivered to the tumors from a ⁶⁰Co source, at the rate of 1 Gy/min with a custom head and body shielding. Tumor-bearing mice received fractionated radiation therapy of 3 Gy a day for 3 consecutive days, 6 h after treatment with either 11C12 or control IgG (5 mg/kg each). Other controls included non-treated animals (vehicle alone), irradiation alone (IR alone) or 11C12 alone. Tissue samples were collected 24 h after the last cycle of treatment and the number of proliferative cells in each treatment condition was measured by Ki67 staining. Proliferative cells are indicated by positive Ki67 staining, a nuclear dark brown staining. Graphic shows the quantification of Ki-67 positive cells in each experimental condition. (FIG. 16B) Combined treatment of H460-derived lung cancer xenografts with 11C12 and IR does not induce apoptosis. Tumor bearing mice were treated as previously described and tissue samples were collected 24 h after the end of treatment. Apoptosis was assessed by TUNEL staining. Significant alterations were not detected in the number of apoptotic cells among the experimental groups. (FIG. 16C) Combined treatment of H460-derived lung cancer xenografts with 11C12 and IR induces cellular senescence in vivo. Tumor bearing mice were treated as previously described; tissue samples were collected 10 days after the last day of treatment, and processed for β-Galactosidase Staining. The staining protocol is designed to detect β-galactosidase activity at pH 6, a known characteristic of senescent cells not found in presenescent, quiescent or immortal cells. The graphic represents the quantification of cells positively stained for β-galactosidase activity.

FIG. 17: Nude rats were inoculated with H460 cancer cells and were enrolled in an imaging study to evaluate antibody distribution in vivo. Experiments were performed when the tumors reached a volume of 300-500 mm³ SPECT/CT images of ¹¹¹In-DTPA-11C12 antibody, or a ¹¹¹In-DTPA-mouse IgG control antibody were acquired 3 h, 24 h, 48 h, 96 h and 144 hours post-injection. Three experimental groups were analysed: ¹¹¹In-DTPA-11C12 injected in naïve (non-tumor bearing) rats (FIG. 17A); In-DTPA-11C12 antibody injected in H460 tumor bearing rats (FIG. 17B—arrows point to tumors) and ¹¹¹In-DTPA-mouse IgG control injected in H460 tumor bearing rats (FIG. 17C—arrows point to tumors). Focused images of tumors are also provided for each time point.

FIG. 18: Analysis of antibody distribution in H460 tumors. FIG. 18A shows the concentration of both 11C12 and IgG control in the tumor as a function of time after injection. FIG. 18B shows the distribution of 11C12 in different regions of the tumor, as compared to the control IgG.

### DETAILED DESCRIPTION OF CERTAIN EMBODIMENTS OF THE INVENTION

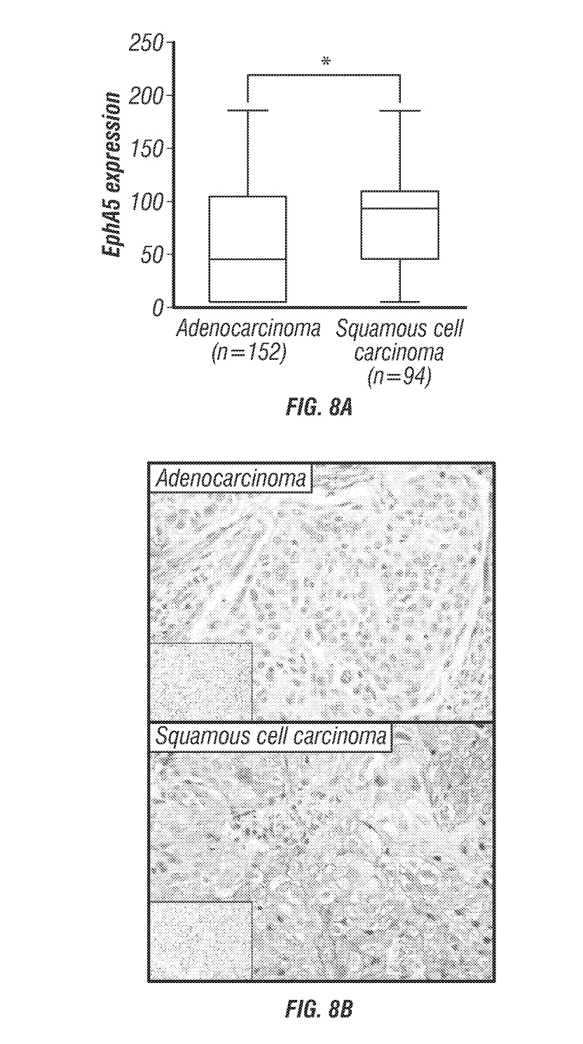

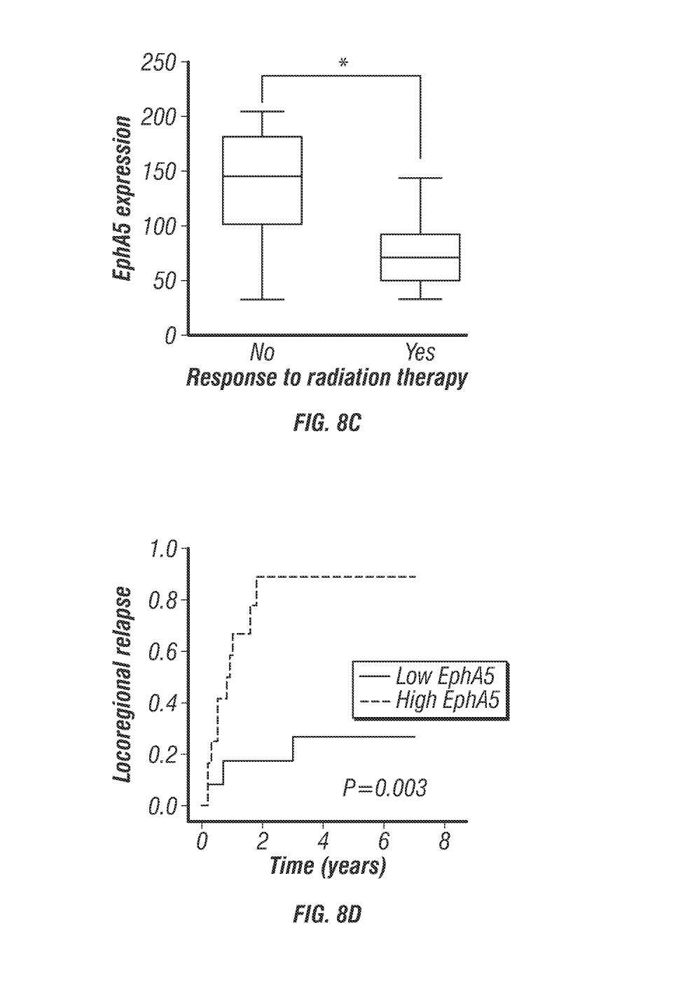

The present invention encompasses the recognition that receptor tyrosine kinases, such as Eph receptors (e.g., EphA5), play an important role in control of cell cycle arrest and in regulation of DNA damage repair. For example, the present inventors have surprisingly found that certain cancers (e.g., lung cancers, cervical cancers, and head and neck cancers) overexpress the receptor tyrosine kinase EphA5, which facilitates DNA damage repair and promotes resistance of the cells to ionizing radiation. Indeed, the present inventors have found that, in response to genotoxic stress, EphA5 is phosphorylated and present in the nucleus of cancer cells. The inventors also found that a direct interaction between EphA5 and activated ATM (phospho-ATM) occurs at sites of DNA damage, which is contemplated to render the cells resistant to ionizing radiation therapy. Analysis of patient samples revealed that elevated EphA5 expression correlated with a poor response to radiation therapy, whereas patients with tumors having low levels of EphA5 were more likely to have a favorable response to such therapy (see, for example, FIG. 8C).

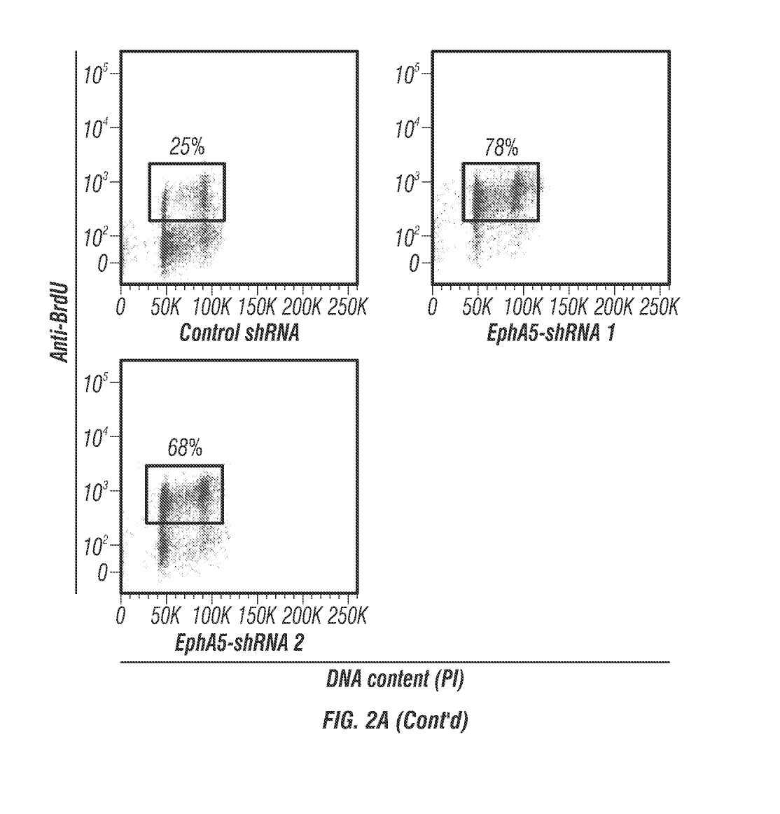

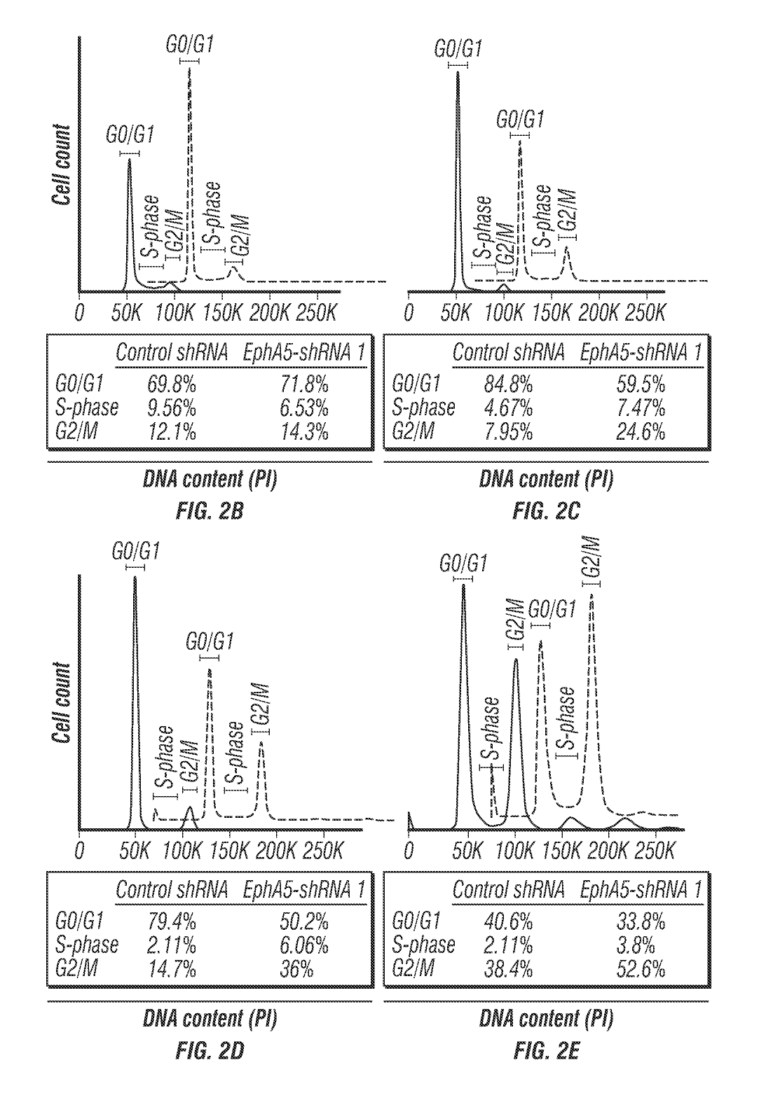

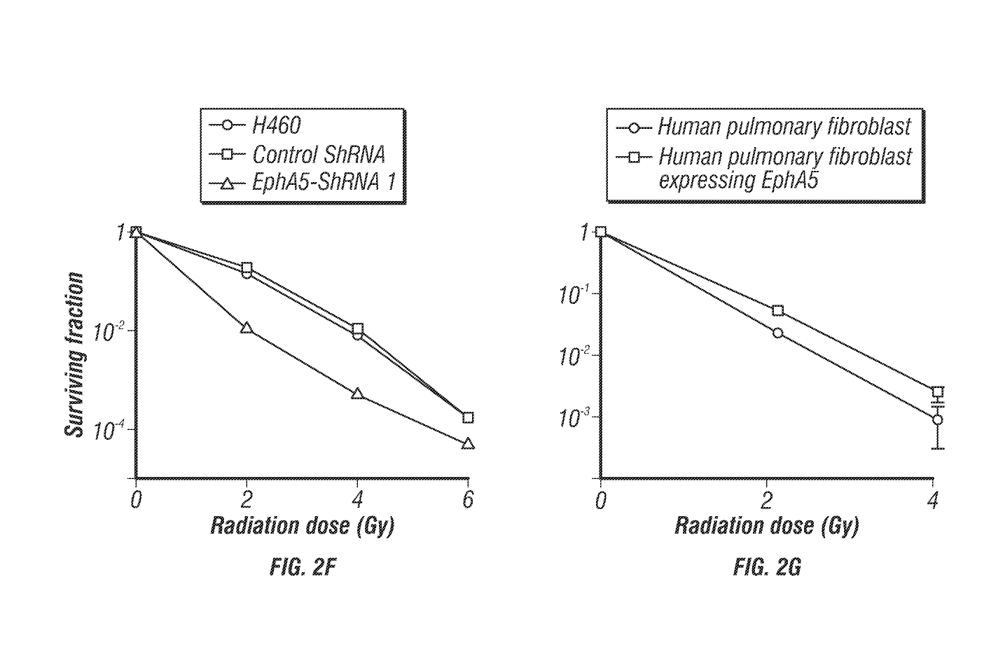

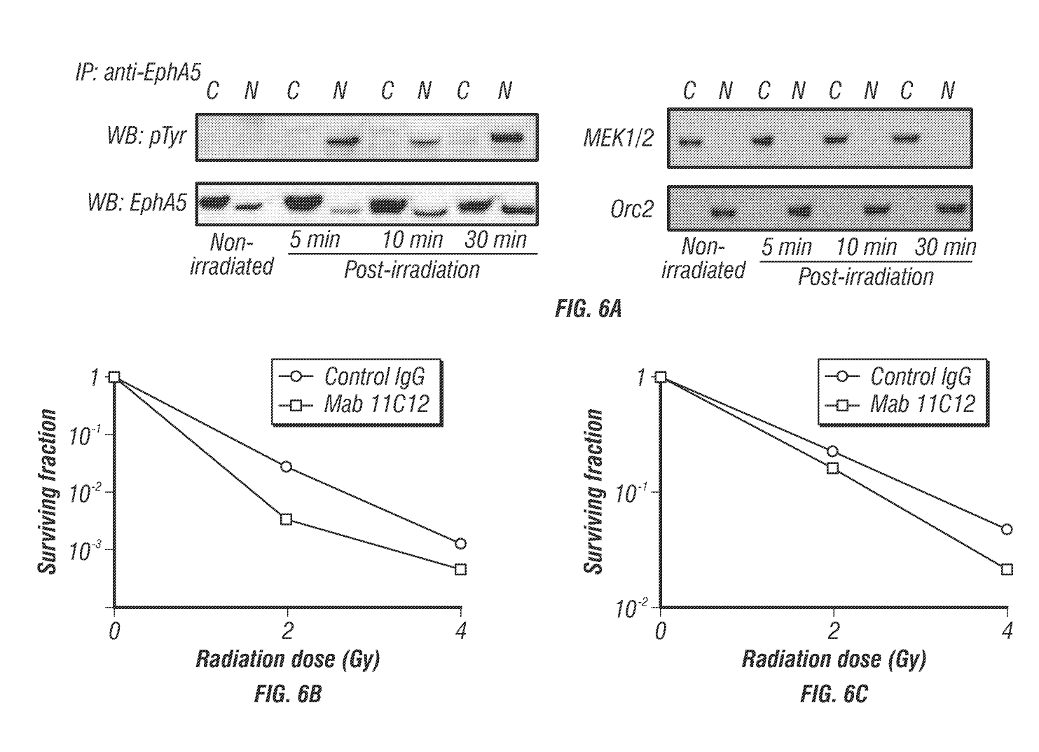

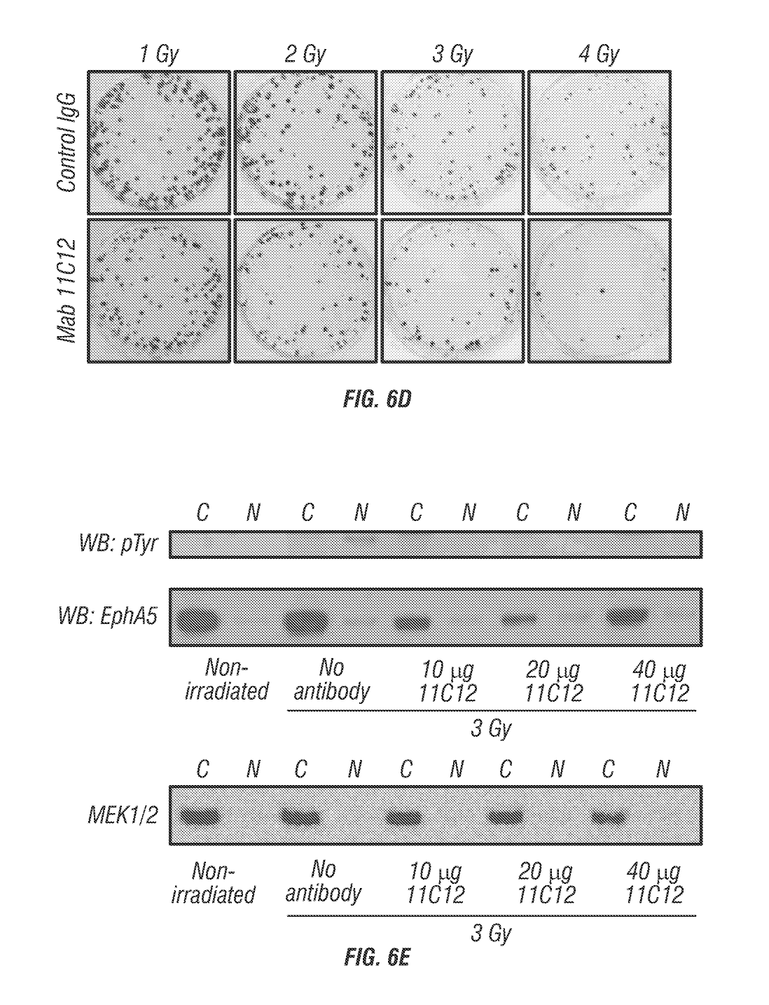

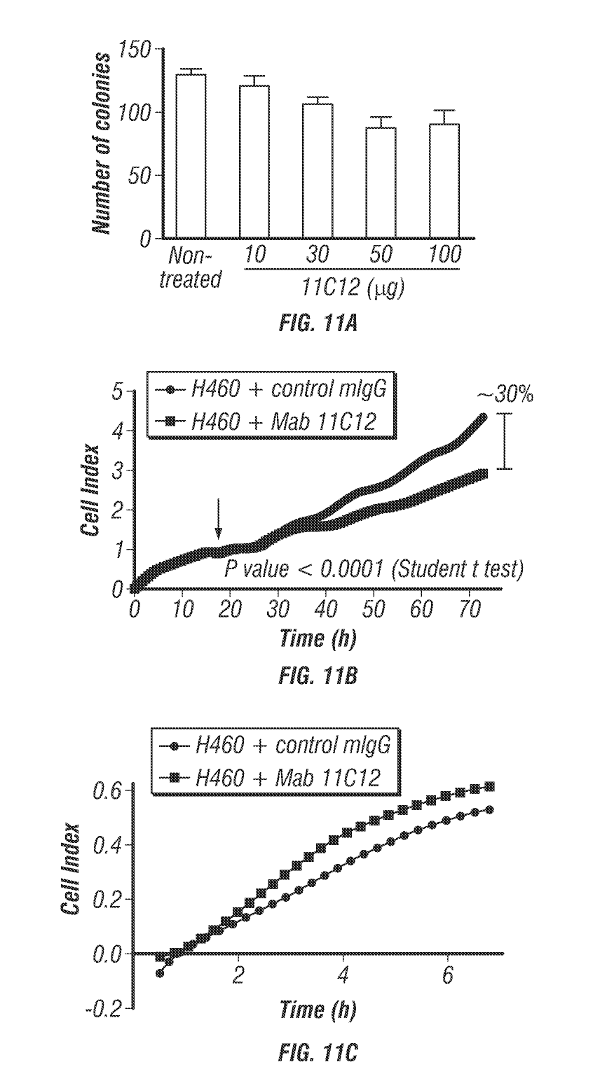

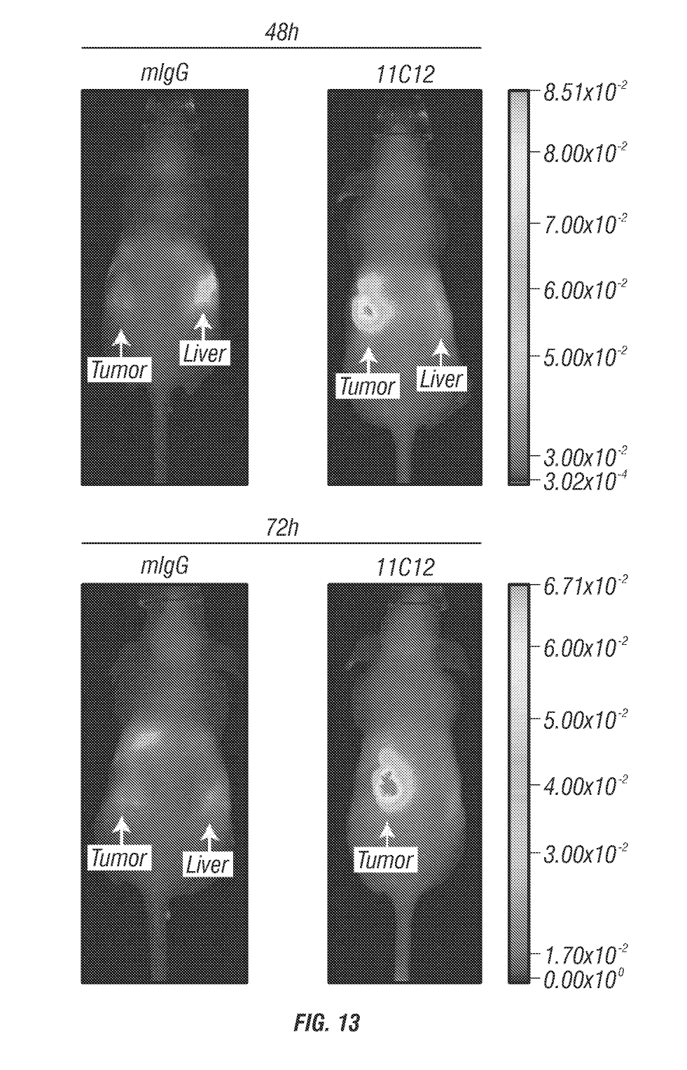

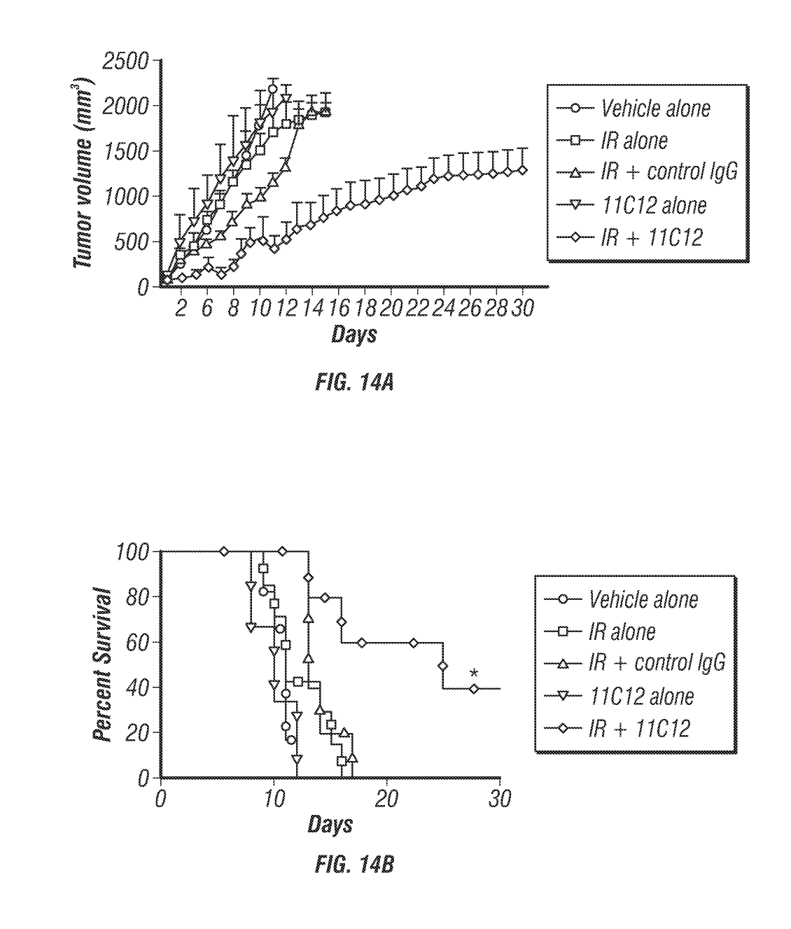

Importantly by modulating EphA5 expression or activation (e.g., phosphorylation and/or nuclear import) in cells, cell proliferation can be slowed and the cells sensitized to the effects of DNA damaging agents. For example, short hairpin RNA (shRNA)-mediated reduction of EphA5 expression sensitized lung cancer cells to killing by ionizing radiation (see FIG. 2F). The inventors also demonstrated that certain anti-EphA5 antibodies can, when applied to cells, block EphA5 phosphorylation, nuclear translocation and binding pATM. Cells contacted with the antibody were likewise rendered sensitive to killing by radiation (FIG. 6B, D). Moreover, even absent radiation application, an anti-EphA5 antibody was able to reduce cancer cell proliferation by nearly ⅓ relative to controls (FIG. 11A, B). In vivo studies further indicate that, when administered to mice bearing human tumor cells, anti-EphA5 antibodies specifically target and accumulate within the tumor tissue (FIG. 13). When such mice were administered anti-EphA5 antibody in conjunction with radiation, tumor growth in the animals was significantly decreased relative to animals administered either agent individually (FIG. 14). Thus, among other things, the inventors have established EphA5 as a target for therapeutic intervention in disorders involving abnormal cell proliferation, such as cancer.

Thus, among other things, the present invention provides compositions and methods for treating a cancer associated with elevated expression and/or activity of receptor tyrosine kinases (e.g., Eph receptors, such as EphA5). In some embodiments, the present invention provides compositions and methods for identifying elevated expression or activity of receptor tyrosine kinases (e.g., Eph receptors, such as EphA5).

### I. DEFINITIONS

Antibody: As used herein, the term “antibody” is intended to include immunoglobulins and fragments thereof which are specifically reactive to the designated protein or peptide, or fragments thereof. Suitable antibodies include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, antibody-like molecules, and antibody fragments. As used herein, the term “antibodies” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. Antibody polypeptides for use herein may be of any type (e.g., IgA, IgD, IgE, IgG, IgM).

Antibody fragment: As used herein, an “antibody fragment” includes a portion of an intact antibody, such as, for example, the antigen-binding or variable region of an antibody. Examples of antibody fragments include Fab, Fab′, F(ab′)2, Fc and Fv fragments; triabodies; tetrabodies; linear antibodies; single-chain antibody molecules; and multi specific antibodies formed from antibody fragments. The term “antibody fragment” also includes any synthetic or genetically engineered protein that acts like an antibody by binding to a specific antigen to form a complex. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region.

Combination therapy: The term “combination therapy”, as used herein, refers to those situations in which two or more different therapeutic agents (e.g., a chemotheraptic, an EphA-targeted agent or a radiation dose) are administered in conjunction either sequentially or essentially simultaneously to a subject. In certain aspects, wherein the combination therapy involves two or more pharmaceutical agents, such agents can be mix into a combined formulation.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

Host: The term “host” is used herein to refer to a system (e.g., a cell, organism, etc.) in which a nucleic acid or polypeptide of interest is present. In some embodiments, a host is a system that expresses a particular polypeptide of interest.

Isolated: The term “isolated”, as used herein, refers to an agent or entity that has either (i) been separated from at least some of the components with which it was associated when initially produced (whether in nature or in an experimental setting); or (ii) produced by the hand of man. Isolated agents or entities may be separated from at least about 10%, at least about 20%, at least about 30%, at least about 40%, at least about 50%, at least about 60%, at least about 70%, at least about 80%, at least about 90%, or more of the other components with which they were initially associated. In some embodiments, isolated agents are more than 90%, 91%, 92%, 93%, 94%, 95%, 96%, 97%, 98%, 99% pure.

Nucleic acid molecule: The term “nucleic acid molecule” is used broadly to mean any polymer of two or more nucleotides, which are linked by a covalent bond such as a phosphodiester bond, a thioester bond, or any of various other bonds known in the art as useful and effective for linking nucleotides. Such nucleic acid molecules can be linear, circular or supercoiled, and can be single stranded or double stranded, e.g. single stranded or double stranded DNA, RNA or DNA/RNA hybrid. In some embodiments, nucleic acid molecules are or include nucleic acid analogs that are less susceptible to degradation by nucleases than are DNA and/or RNA. For example, RNA molecules containing 2′-O-methylpurine substitutions on the ribose residues and short phosphorothioate caps at the 3′- and 5′-ends exhibit enhanced resistance to nucleases (Green et al., Chem. Biol., 2:683-695 (1995), which is incorporated herein by reference). Similarly, RNA containing 2′-amino-2′-deoxypyrimidines or 2′-fluro-2′-deoxypyrimidines is less susceptible to nuclease activity (Pagratis et al., Nature Biotechnol., 15:68-73 (1997), which is incorporated herein by reference). Furthermore, L-RNA, which is a stereoisomer of naturally occurring D-RNA, is resistant to nuclease activity (Nolte et al., Nature Biotechnol., 14:1116-1119 (1996); Klobmann et al., Nature Biotechnol., 14:1112-1115 (1996); each of which is incorporated herein by reference). Such RNA molecules and methods of producing them are well known in the art and can be considered to be routine (see Eaton and Piekern, Ann. Rev. Biochem., 64:837-863 (1995), which is incorporated herein by reference). DNA molecules containing phosphorothioate linked oligodeoxynucleotides are nuclease resistant (Reed et al., Cancer Res. 50:6565-6570 (1990), which is incorporated herein by reference). Phosphorothioate-3′ hydroxypropylamine modification of the phosphodiester bond also reduces the susceptibility of a DNA molecule to nuclease degradation (see Tam et al., Nucl. Acids Res., 22:977-986 (1994), which is incorporated herein by reference).

Obtaining: Many methodologies described herein include a step of “obtaining” Those of ordinary skill in the art, reading the present specification, will appreciate that such “obtaining” can encompass both receiving information or materials from a source (e.g., a laboratory report) and directly determining information (e.g., by manipulation of a physical sample). In some embodiments, a obtaining involves consideration and/or manipulation of data or information, for example utilizing a computer or other processing unit adapted to perform a relevant analysis.

Organ or Tissue: As used herein, the terms “organ or tissue” and “selected organ or tissue” are used in the broadest sense to mean an organ or tissue in or from a body. In some embodiments, an organ or tissue has a pathology, for example, lung containing lung tumors, whether primary or metastatic lesions. In some embodiments, an organ or tissue is normal. The term “control organ or tissue” is used to mean an organ or tissue other than a selected organ or tissue of interest. In some embodiments, a control organ or tissue is characterized by the inability of a ligand-encoding phage to home to the control organ or tissue and, therefore, is useful for identifying selective binding of a molecule to a selected organ or tissue.

Polypeptide: A “polypeptide”, generally speaking, is a string of at least two amino acids attached to one another by a peptide bond. In some embodiments, a polypeptide includes at least 3-5 amino acids, each of which is attached to others by way of at least one peptide bond. Those of ordinary skill in the art will appreciate that, in some embodiments, polypeptides include one or more “non-natural” amino acids or other entities that nonetheless are capable of integrating into a polypeptide chain. In some embodiments, a polypeptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues. A polypeptide as described herein may be a member of a polypeptide family or class. As will be understood by those skilled in the art, polypeptide families or classes are defined by shared structural elements (e.g., preservation of one or more characteristic sequence elements, which may include sets of identical or similar residues separated from one another by defined distances, and/or a specified degree of overall sequence identity. In some embodiments, members of a polypeptide family or class share an overall sequence identity of at least about 40%, at least about 50%, at least about 60%, at least about 70%, at least about 80%, at least about 90%, at least about 90%, at least about 91%, at least about 92%, at least about 93%, at least about 94%, at least about 95%, at least about 96%, at least about 97%, at least about 98%, at least about 99% or more. In some embodiments, members of a polypeptide family or class shoe substantial sequence identity to one another. Alternatively or additionally, in some embodiments, members of a polypeptide family or class have similar lengths, typically not differing from each other by more than 50%, more than 45%, more than 40%, more than 35%, more than 30%, more than 25%, more than 20%, more than 15%, more than 10%, more than 5%, more than 4%, more than 3%, more than 2%, more than 1%, or less.

Sample: As used herein, the term “sample” refers to a cell, tissue, organ or portion thereof that is isolated from a body. It will be appreciated that a sample may be or comprise a single cell or a plurality of cells. In some embodiments, a sample is or comprises a histologic section or a specimen obtained by biopsy (e.g., surgical biopsy); in some embodiments, a sample is or comprises cells that are or have been placed in or adapted to tissue culture. In some embodiments, a sample is a specimen obtained from a dead body (e.g., by autopsy). In some embodiments, the sample is or comprises an intact organ or tissue. In some embodiments, the sample is or comprises circulating cells, such as circulating tumor cells.

Sample processing: As used herein, the term “sample processing” generally refers to various steps that may be accomplished to prepare a sample for quantification. In some embodiments, crude sample (e.g., whole tissue, homogenized tissue, etc.) is prepared. In some embodiments, purified or highly purified sample is prepared.

Specificity: As is known in the art, “specificity” is a measure of the ability of a particular ligand (e.g., a ligand encoded by a phage) to distinguish its binding partner (e.g., a target tissue, or organ of interest) from other potential binding partners (e.g., a control tissue or organ).

Subject: As used herein, the terms “subject,” “individual” or “patient” refer to a human or a non-human mammalian subject. In some embodiments, a subject is a non-human primate. In some embodiments, the subject is a dog, cat, goat, horse, pig, mouse, rabbit, or the like. In some embodiments, a subject is a human. In some embodiments, a human subject is a patient having a surgical tumor resection or a surgical biopsy. In some embodiments, a human subject is a patient suffering from brain death or trauma. In some embodiments, a human subject is an end-of-life patient.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. Alternatively, homologous residues may be non-identical residues that share one or more structural and/or functional characteristics. For example, as is well known by those of ordinary skill in the art, certain amino acids are typically classified as “hydrophobic” or “hydrophilic” amino acids, and/or as having “polar” or “non-polar” side chains In some embodiments, substitution of one amino acid for another of the same type is considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic acid | Asp | D | polar | negative | −3.5 |
| Cysteine | Cys | C | nonpolar | neutral | 2.5 |
| Glutamic acid | Glu | E | polar | negative | −3.5 |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999; all of the foregoing of which are incorporated herein by reference. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999; all of the foregoing of which are incorporated herein by reference. In addition to identifying identical sequences, the programs mentioned above typically provide an indication of the degree of identity. In some embodiments, two sequences are considered to be substantially identical if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are identical over a relevant stretch of residues. In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Therapeutic agent: As used herein, the phrase “therapeutic agent” refers to any agent that elicits a desired biological or pharmacological effect.

Treatment: As used herein, the term “treatment” refers to any method used to alleviate, delay onset, reduce severity or incidence, or yield prophylaxis of one or more symptoms or aspects of a disease, disorder, or condition. For the purposes of the present invention, treatment can be administered before, during, and/or after the onset of symptoms.

Unit dose form: As used herein, the term “unit dose form” refers to a physically discrete unit of a therapeutic agent for treatment of a patient. Each unit contains a predetermined quantity of active material calculated to produce the desired effect. It will be understood, however, that the total dosage of the composition will be decided by the attending physician within the scope of sound medical judgment.

As used in this application, the terms “about” and “approximately” are used as equivalents. Any numerals used in this application with or without about/approximately are meant to cover any normal fluctuations appreciated by one of ordinary skill in the relevant art.

### II. EPHA

Eph receptor tyrosine kinases and their ligands (ephrin) regulate a wide range of cell contact-dependent signaling that can effect cell proliferation, migration, morphology, adhesion, and invasion (Pitulescu et al. Genes & Dev., 24:2480-2492, 2010). This can occur through Eph signaling which alters the actin cytoskeleton organization and integrins and intercellular adhesion molecules processes. Eph-ephrin interactions are important for many biological roles including axon growth and maturation, cell positioning in the gastoinestinal tract, blood vessel morphogenesis and angiogenic sprouting, insulin secretion, bone remodeling, and immune function.

Eph receptors are typically divided into a globular ligand-binding domain, a cysteine-rich region, and two fibronectin type III repeats in the extracellular region, a short transmembrane region with several conserved tyrosine residues and the tyrosine kinase domain, a sterile a motif (SAM) protein-protein interaction domain, and a C-terminal PDZ-binding motif in the intracellular region. Eph receptors are divided into two classes, EphA and EphB. Likewise, Eph ligands (ephrins), which are also cell-surface associated proteins, divided into two classes, GPI-anchored ephrin-A and transmembrane ephrin-B. Ephrin-B molecules contain a cytoplasmic domain with several highly conserved tyrosine phosphorylation sites and a C-terminal PDZ motif Generally, EphA receptors bind to ephrin-A, and EphB receptors bind to ephrin-B, but cross-signaling can occur. Eph and ephrins are capable of bi-directional signaling through trans interactions, though interactions in cis (i.e., between molecules expressed in the same cell) appear to inhibit receptor activation.

As used herein the term “EphA” refers to EphA receptors in general, such as human EphA1, EphA2, EphA3, EphA4, EphA5, EphA6, EphA7, EphA8 or EphA9. Likewise, ephrin ligands can be referred to generally as ephrinA ligands which encompass human ephrinA1, ephrinA3, ephrinA4, ephrinA5 and ephrinA6. In certain embodiments reference is made to specific EphA molecules, such as EphA5 (NCBI accession No. NM_{—}182472) which is provided as SEQ ID NO:13.

Certain aspects of the embodiments concern obtaining or determining an EphA expression. As used herein obtaining an expression encompasses quantifying an absolute or relative DNA (e.g., genomic copy number), mRNA or protein expression level in a cell or sample. However, obtaining an EphA expression may also encompasses obtaining a sequence of an EphA (e.g., to determine mutations in the sequence relative to a control). Thus, obtaining an expression can involve sequencing, PCR amplification or hybridization to determine DNA or RNA expression or an immunoassay (e.g., Western blot, ELISA or immunofluorescence), ligand binding assay or mass spectroscopy to determine protein expression. some aspects, EphA expression is determined in vivo in a subject (e.g., by using radioisotope or fluorescently labeled antibody or Eph ligand).

Aspects of the embodiments also concern obtaining or determining an EphA activity. As used herein an activity can be determined by any method for assessing EphA receptor activation including, but not limited to, determining EphA kinase activity (e.g., by a kinase assay), EphA phosphorylation level (e.g., by use of phosphorylation specific antibody), EphA binding to a signaling partner (e.g., p-ATM) or EphA nuclear localization.

### III. EPHA-TARGETING AGENTS

Among other things, the present invention provides compositions and methods for treating a cancer associated with elevated expression and/or activity of receptor tyrosine kinases (e.g., Eph receptors, such as EphA5). It will be appreciated that any type of cancer may be associated with elevated expression and/or activity of receptor tyrosine kinases and thus, be targeted by an EphA targeting agent in accordance with the present invention. For example, in some embodiments, a cancer associated with elevated expression and/or activity of receptor typrosine kinases (e.g., Eph receptors, such as EphA5) is squamous cell carcinoma, head and neck cancer, adenocarcinoma, pancreatic cancer, lung cancer, cervical carcinoma, ovarian cancer or prostate cancer, among others.

Certain embodiments of the invention concern agents that target EphA (e.g., EphA5). Particular, embodiments described herein concern agents that are effective at reducing expression of EphA in cell and/or reducing EphA activity in a cell. For example, EphA expression may be reduced by targeting the protein or coding mRNA for degradation or by blocking EphA transcription, mRNA transport or translation. Likewise, EphA activity can be reduced by blocking or reducing its interaction with a ligand molecule (e.g., an Ephrin molecule), by blocking or reducing EphA phosphorylation, by reducing EphA transport to the cell nucleus or by reducing EphA interaction binding partners involved in its activity in the cell (e.g., pATM). Thus, in certain aspects an EphA-targeting agent is a molecule that binds to an EphA protein, such as a EphA-binding polypeptide (e.g., an anticalin), antibody or a nucleic acid (e.g., an aptamer). Such targeting agents can be used to reduce EphA expression for example by targeting the polypeptide for degradation. In other aspects the EphA binding molecules can reduce EphA activity by altering its three-dimensional structure, directly blocking sites of phosphorylation, or by displacing or competing with EphA ligands or binding partners. Thus, such binding agents can bind to either intracellular or extracellular portions of an EphA polypeptide. In some aspects, EphA-binding agents have a specificity for an extracellular portion of EphA such that the agents do not need to traverse the cell membrane to affect their activity. Certain non-limiting and exemplary EphA binding molecules are discussed in detail below.

A. Immunological Reagents

In particular embodiments of the invention, immunological reagents are employed. For example, antibodies may be utilized to bind EphA (e.g., EphA5), to target EphA-expressing cells, reduce EphA expression or to reduce EphA activity in a cell. In some embodiments, antibodies to EphA are employed in diagnostic aspects of the invention, such as for detecting the presence of EphA on or in a cell, in a cell nucleus or for detecting EphA phosphorylation. Antibodies may be of any suitable kind, although in some embodiments they comprise monoclonal antibodies that compete with a ligand or the 11C12 antibody for binding to EphA5. For example, an anti-EphA5 antibody may comprise 1 or more (or all) of the CDRs of the 11C12 antibody (see, e.g., SEQ ID NOs:11 and 12, which provide the VH and VL amino acid sequences).

VH Sequence:

CDR1: aa31-aa35. Seq:
(SEQ ID NO: 14)
TFGIH
CDR2: aa50-aa66. Seq:
(SEQ ID NO: 15)
YISGASTTIYYADTVKG
CDR3: aa99-aa109. Seq:
(SEQ ID NO: 16)
YGTSFPYGLDY

VL Sequence:

CDR1: aa24-aa38. Seq:
(SEQ ID NO: 17)
KASQSVDYDGDSYMN
CDR2: aa54-aa60. Seq:
(SEQ ID NO: 18)
GASNLES
CDR3: aa93-aa99. Seq:
(SEQ ID NO: 19)
QQSNEDP

1. Antibodies

In certain aspects of the invention, one or more antibodies may be obtained or produced which have a specificity for an EphA. These antibodies may be used in various diagnostic or therapeutic applications described herein. A number of antibodies with specificity to EphA are commercially available. For example, commercially available anti-EphA5 antibodies for use according to the instant invention include, but are not limited to, the monoclonal antibodies 6F4, 5C2, 5C3, 5C8 (each of which was raised against amino acids 234-333 of the human EphA5 protein) 8B10B1 and 8B10F5 (each raised against amino acids 620-744 of the human EphA5 protein) all of which are available from Novus Biologics. A phosphorylation specific anti-EphA5, which recognized EphA5 that is phosphorylated at tyrosine 833 is also available from Epitomics (Cat.#: 5252-1). Multiple suppliers also provided commercial polyclonal anti-EphA5 antisera which can be used in accordance with the embodiments. For example, such polyclonal antisera is available from Aviva Systems Biology (cat# ARP58460_P050), Sigma-Aldrich (SAB2100689), Santa Cruz Biotechnology, (clone L15), abcam (ab5398) and Novus Biologics (NBP1-53105).

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)2, single domain antibodies (DABs), Fv, scFv (single chain Fv), and polypeptides with antibody CDRs, scaffolding domains that display the CDRs (e.g., anticalins) or a nanobody. For example, the antibody can be a VHH (i.e., an antigen-specific VHH) antibody that comprises only a heavy chain. For example, such antibody molecules can be derived from a llama or other camelid antibody (e.g., a camelid IgG2 or IgG3, or a CDR-displaying frame from such camelid Ig) or from a shark antibody. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

“Mini-antibodies” or “minibodies” are also contemplated for use with the present invention. Minibodies are sFv polypeptide chains which include oligomerization domains at their C-termini, separated from the sFv by a hinge region. Pack et al. (1992) Biochem 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992) Biochem 31:1579-1584; Cumber et al. (1992) J Immunology 149B:120-126.

Antibody-like binding peptidomimetics are also contemplated in the present invention. Liu et al. Cell Mol Biol (Noisy-le-grand). 2003 March; 49(2):209-16 describe “antibody like binding peptidomimetics” (ABiPs), which are peptides that act as pared-down antibodies and have certain advantages of longer serum half-life as well as less cumbersome synthesis methods. Likewise, in some aspects, antibody-like molecules are cyclic or bicyclic peptides. For example, methods for isolating antigen-binding bicyclic peptides (e.g., by phage display) and for using the such peptides are provided in U.S. Patent Publn. No. 20100317547, incorporated herein by reference.

Monoclonal antibodies (MAbs) are recognized to have certain advantages, e.g., reproducibility and large-scale production. Embodiments of the invention provide monoclonal antibodies of the human, murine, monkey, rat, hamster, rabbit and chicken origin. Due to the ease of preparation and ready availability of reagents, murine monoclonal antibodies will often be preferred.

“Humanized” antibodies are also contemplated, as are chimeric antibodies from mouse, rat, or other species, bearing human constant and/or variable region domains, bispecific antibodies, recombinant and engineered antibodies and fragments thereof. As used herein, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor”. A “humanized antibody” is an antibody comprising a humanized light chain and a humanized heavy chain immunoglobulin.

i. Methods for Generating Antibodies

Methods for generating antibodies (e.g., polyclonal and/or monoclonal antibodies) are known in the art.

Briefly, a polyclonal antibody is prepared by immunizing an animal with a EphA polypeptide a portion thereof in accordance with the present invention and collecting antisera from that immunized animal. For example, a portions of an EphA (such as EphA5), including but not limited to, the extracellular domain, the ephrin binding domain, the pATM binding domain, the kinase domain or regions including sites of phosphorylation (either phosphorylated or not) can be used as an immunogen.

A wide range of animal species can be used for the production of antisera. Typically the animal used for production of antisera is a rabbit, a mouse, a rat, a hamster, a guinea pig or a goat. The choice of animal may be decided upon the ease of manipulation, costs or the desired amount of sera, as would be known to one of skill in the art. It will be appreciated that antibodies of the embodiments can also be produced transgenically through the generation of a mammal or plant that is transgenic for the immunoglobulin heavy and light chain sequences of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957.

As is also well known in the art, the immunogenicity of a particular immunogen composition can be enhanced by the use of non-specific stimulators of the immune response, known as adjuvants. Suitable adjuvants include any acceptable immunostimulatory compounds, such as cytokines, chemokines, cofactors, toxins, plasmodia, synthetic compositions or vectors encoding such adjuvants.

Adjuvants that may be used in accordance with the present embodiments include, but are not limited to, IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion is also contemplated. MHC antigens may even be used. Exemplary, adjuvants may include complete Freund's adjuvant (a non-specific stimulator of the immune response containing killed Mycobacterium tuberculosis), incomplete Freund's adjuvants and/or aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as γ-interferon, IL-2, or IL-12 or genes encoding proteins involved in immune helper functions, such as B-7.

The amount of immunogen composition used in the production of antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

For production of rabbit polyclonal antibodies, the animal can be bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography, among others.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells.

The methods for generating monoclonal antibodies (MAbs) generally begin along the same lines as those for preparing polyclonal antibodies. In some embodiments, Rodents such as mice and rats are used in generating monoclonal antibodies. In some embodiments, rabbit, sheep or frog cells are used in generating monoclonal antibodies. The use of rats is well known and may provide certain advantages (Goding, 1986, pp. 60 61). Mice (e.g., BALB/c mice) are routinely used and generally give a high percentage of stable fusions.

The animals are injected with antigen, generally as described above. The antigen may be mixed with adjuvant, such as Freund's complete or incomplete adjuvant. Booster administrations with the same antigen or DNA encoding the antigen may occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Generally, spleen cells are a rich source of antibody-producing cells that are in the dividing plasmablast stage. Typically, peripheral blood cells may be readily obtained, as peripheral blood is easily accessible.

In some embodiments, a panel of animals will have been immunized and the spleen of an animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody producing B lymphocytes from the immunized animal are then fused with cells of an immortal myeloma cell, generally one of the same species as the animal that was immunized Myeloma cell lines suited for use in hybridoma producing fusion procedures preferably are non antibody producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65 66, 1986; Campbell, pp. 75 83, 1984). For example, where the immunized animal is a mouse, one may use P3 X63/Ag8, X63 Ag8.653, NS1/1.Ag 4 1, Sp210 Ag14, FO, NSO/U, MPC 11, MPC11 X45 GTG 1.7 and S194/5XX0 Bu1; for rats, one may use R210.RCY3, Y3 Ag 1.2.3, IR983F and 4B210; and U 266, GM1500 GRG2, LICR LON HMy2 and UC729 6 are all useful in connection with human cell fusions. See Yoo et al., J Immunol Methods. 2002 Mar. 1; 261(1-2):1-20, for a discussion of myeloma expression systems.

One murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8 azaguanine resistant mouse murine myeloma SP2/0 non producer cell line.

Methods for generating hybrids of antibody producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al., (1977). The use of electrically induced fusion methods is also appropriate (Goding pp. 71 74, 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

Further, expression of antibodies of the invention (or other moieties therefrom) from production cell lines can be enhanced using a number of known techniques. For example, the glutamine synthetase and DHFR gene expression systems are common approaches for enhancing expression under certain conditions. High expressing cell clones can be identified using conventional techniques, such as limited dilution cloning and Microdrop technology. The GS system is discussed in whole or part in connection with European Patent Nos. 0 216 846, 0 256 055, and 0 323 997 and European Patent Application No. 89303964.4.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonal antibodies. In one embodiment, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies. Target-binding single domain antibodies can also be isolated by use of display libraries, see for example, U.S. Patent Appln. No. 20110183863, incorporated herein by reference. Ribosome expression libraries for isolation of target-bind Ig coding sequences are also described in U.S. Patent Appln. No. 20040161748; 20070299246 and 20080293591, each incorporated herein by reference.

Another embodiment of the invention for producing antibodies according to the present invention is found in U.S. Pat. No. 6,091,001, which describes methods to produce a cell expressing an antibody from a genomic sequence of the cell comprising a modified immunoglobulin locus using Cre-mediated site-specific recombination is disclosed. The method involves first transfecting an antibody-producing cell with a homology-targeting vector comprising a lox site and a targeting sequence homologous to a first DNA sequence adjacent to the region of the immunoglobulin loci of the genomic sequence which is to be converted to a modified region, so the first lox site is inserted into the genomic sequence via site-specific homologous recombination. Then the cell is transfected with a lox-targeting vector comprising a second lox site suitable for Cre-mediated recombination with the integrated lox site and a modifying sequence to convert the region of the immunoglobulin loci to the modified region. This conversion is performed by interacting the lox sites with Cre in vivo, so that the modifying sequence inserts into the genomic sequence via Cre-mediated site-specific recombination of the lox sites.

Alternatively, monoclonal antibody fragments encompassed by the present invention can be synthesized using an automated peptide synthesizer, or by expression of full-length gene or of gene fragments in E. coli.

ii. Antibody Conjugates

The present invention provides antibodies against EphA proteins, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins (e.g., TNF alpha or gelanin), anti-tumor agents, therapeutic enzymes, radiolabeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin. For example, an antibody can be conjugated to a maytansinoid (e.g., maytansinol or the DM1 maytansinoid, see, U.S. Pat. Nos. 5,208,020; 6,333,410; and 7,276,497), auriculin, calicheamicin, duocarmicin or tubulysin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might use astatine211, 14carbon, 51chromium, 36chlorine, 57cobalt, 58cobalt, copper67, 152Eu, gallium67, 3hydrogen, iodine123, iodine125, iodine131, indium111, 59iron, 32phosphorus, radium223, rhenium186, rhenium188, 75selenium, 35sulphur, technicium99m, thorium227 and/or yttrium90. 125O is often used in certain embodiments, and technicium99m and/or indium111 are also often used due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium99m by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red, among others.

Antibody conjugates contemplated in the present invention include those intended primarily for use in vitro, where the antibody is linked to a secondary binding ligand and/or to an enzyme (an enzyme tag) that will generate a colored product upon contact with a chromogenic substrate. Examples of suitable enzymes include, but are not limited to, urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In some embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

In some embodiments of the invention, anti-EphA antibodies are linked to semiconductor nanocrystals such as those described in U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999). In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AlS, AlP, AlSb, PbS, PbSe, Ge and Si and ternary and quaternary mixtures thereof. Methods for linking semiconductor nanocrystals to antibodies are described in U.S. Pat. Nos. 6,630,307 and 6,274,323.

2. Antibody-Like Molecules

Alternative scaffolds for antigen binding peptides, are also available and can be used to generate EphA-binding molecules in accordance with the embodiments.

Generally, a person skilled in the art knows how to determine the type of protein scaffold on which to graft at least one of the CDRs arising from the original antibody. More particularly, it is known that to be selected such scaffolds must meet the greatest number of criteria as follows (Skerra A., J. Mol. Recogn., 2000, 13:167-187): good phylogenetic conservation; known three-dimensional structure (as, for example, by crystallography, NMR spectroscopy or any other technique known to a person skilled in the art); small size; few or no post-transcriptional modifications; and/or easy to produce, express and purify.

The origin of such protein scaffolds can be, but is not limited to, the structures selected among: fibronectin and preferentially fibronectin type III domain 10, lipocalin, anticalin (Skerra A., J. Biotechnol., 2001, 74(4):257-75), protein Z arising from domain B of protein A of Staphylococcus aureus, thioredoxin A or proteins with a repeated motif such as the “ankyrin repeat” (Kohl et al., PNAS, 2003, vol. 100, No. 4, 1700-1705), the “armadillo repeat”, the “leucine-rich repeat” and the “tetratricopeptide repeat”. For example, anticalins or lipocalin derivatives are a type of binding proteins that have affinities and specificities for various target molecules and can be used as EphA binding molecules. Such proteins are described in US Patent Publication Nos. 20100285564, 20060058510, 20060088908, 20050106660, and PCT Publication No. WO2006/056464, incorporated herein by reference.

Scaffolds derived from toxins such as, for example, toxins from scorpions, insects, plants, mollusks, etc., and the protein inhibitors of neuronal NO synthase (PIN) may also be used in certain aspects.

B. EphA-Targeted Nucleic Acids

As mentioned above, the present invention contemplates the use of one or more inhibitory nucleic acid for reducing expression and/or activation of an EphA protein. Examples of an inhibitory nucleic acid include but are not limited to molecules targeted to an EphA nucleic acid sequence, such as an siRNA (small interfering RNA), short hairpin RNA (shRNA), double-stranded RNA, an antisense oligonucleotide, a ribozyme and molecules targeted to a EphA polypeptide such as an aptamer.

1. EphA Nucleic Acid Targeted Molecules

An inhibitory nucleic acid may inhibit the transcription of a gene or prevent the translation of a EphA gene transcript in a cell. An inhibitory nucleic acid may be from 16 to 1000 nucleotides long, and in certain embodiments from 18 to 100 nucleotides long. In certain embodiments, the inhibitory nucleic acid is an isolated nucleic acid that binds or hybridizes to a EphA nucleotide sequence such as the EphA5 coding sequence of SEQ ID NO:13.

Inhibitory nucleic acids are well known in the art. For example, siRNA, shRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Since the discovery of RNAi by Fire and colleagues in 1998, the biochemical mechanisms have been rapidly characterized. Double stranded RNA (dsRNA) is cleaved by Dicer, which is an RNAase III family ribonuclease. This process yields siRNAs of ˜21 nucleotides in length. These siRNAs are incorporated into a multiprotein RNA-induced silencing complex (RISC) that is guided to target mRNA. RISC cleaves the target mRNA in the middle of the complementary region. In mammalian cells, the related microRNAs (miRNAs) are found that are short RNA fragments (˜22 nucleotides). MiRNAs are generated after Dicer-mediated cleavage of longer (˜70 nucleotide) precursors with imperfect hairpin RNA structures. The miRNA is incorporated into a miRNA-protein complex (miRNP), which leads to translational repression of target mRNA.

In designing RNAi there are several factors that need to be considered such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Particularly the siRNA exhibits greater than 80, 85, 90, 95, 98% or even 100% identity between the sequence of the siRNA and a portion of a EphA nucleotide sequence. Sequences less than about 80% identical to the target gene are substantially less effective. Thus, the greater identity between the siRNA and the EphA gene to be inhibited, the less likely expression of unrelated genes will be affected.

In addition, the size of the siRNA is an important consideration. In some embodiments, the present invention relates to siRNA molecules that include at least about 19-25 nucleotides, and are able to modulate EphA gene expression. In the context of the present invention, the siRNA is particularly less than 500, 200, 100, 50, 25, 24, 23 or 22 nucleotides in length. In some embodiments, the siRNA is from about 25 nucleotides to about 35 nucleotides or from about 19 nucleotides to about 25 nucleotides in length.

To improve the effectiveness of siRNA-mediated gene silencing, guidelines for selection of target sites on mRNA have been developed for optimal design of siRNA (Soutschek et al., 2004; Wadhwa et al., 2004). These strategies may allow for rational approaches for selecting siRNA sequences to achieve maximal gene knockdown. To facilitate the entry of siRNA into cells and tissues, a variety of vectors including plasmids and viral vectors such as adenovirus, lentivirus, and retrovirus have been used (Wadhwa et al., 2004).

Within an inhibitory nucleic acid, the components of a nucleic acid need not be of the same type or homogenous throughout (e.g., an inhibitory nucleic acid may comprise a nucleotide and a nucleic acid or nucleotide analog). Typically, an inhibitory nucleic acid form a double-stranded structure; the double-stranded structure may result from two separate nucleic acids that are partially or completely complementary. In certain embodiments of the present invention, the inhibitory nucleic acid may comprise only a single nucleic acid (polynucleotide) or nucleic acid analog and form a double-stranded structure by complementing with itself (e.g., forming a hairpin loop). The double-stranded structure of the inhibitory nucleic acid may comprise 16-500 or more contiguous nucleobases, including all ranges therebetween. The inhibitory nucleic acid may comprise 17 to 35 contiguous nucleobases, more particularly 18 to 30 contiguous nucleobases, more particularly 19 to 25 nucleobases, more particularly 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

siRNA can be obtained from commercial sources, natural sources, or can be synthesized using any of a number of techniques well-known to those of ordinary skill in the art. For example, commercial sources of predesigned siRNA include Invitrogen's Stealth™ Select technology (Carlsbad, Calif.), Ambion® (Austin, Tex.), and Qiagen® (Valencia, Calif.). An inhibitory nucleic acid that can be applied in the compositions and methods of the present invention may be any nucleic acid sequence that has been found by any source to be a validated downregulator of a EphA.

In some embodiments, the invention features an isolated siRNA molecule of at least 19 nucleotides, having at least one strand that is substantially complementary to at least ten but no more than thirty consecutive nucleotides of a nucleic acid that encodes EphA, and that reduces the expression of EphA. In some embodiments of the present invention, the siRNA molecule has at least one strand that is substantially complementary to at least ten but no more than thirty consecutive nucleotides of the mRNA that encodes EphA.

In some embodiments, the siRNA molecule is at least 75, 80, 85, or 90% homologous, particularly at least 95%, 99%, or 100% similar or identical, or any percentages in between the foregoing (e.g., the invention contemplates 75% and greater, 80% and greater, 85% and greater, and so on, and said ranges are intended to include all whole numbers in between), to at least 10 contiguous nucleotides of any of the nucleic acid sequences encoding a full-length EphA protein.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Publication 2004/0019001 and U.S. Pat. No. 6,673,611 (each of which is incorporated by reference in its entirety). Collectively, all such altered nucleic acids or RNAs described above are referred to as modified siRNAs.

In some embodiments, RNAi is capable of decreasing the expression of EphA (e.g., EphA5) by at least 10%, at least 20%, at least 30%, or at least 40%, at least 50%, at least 60%, or at least 70%, at least 75%, at least 80%, at least 90%, at least 95% or more or any ranges in between the foregoing.

Certain embodiments of the present invention pertain to methods of inhibiting expression of a gene encoding EphA in a cell by introduction of inhibitory nucleic acids into the cell. Introduction of siRNA into cells can be achieved by methods known in the art, including for example, microinjection, electroporation, or transfection of a vector comprising a nucleic acid from which the siRNA can be transcribed. Alternatively, a siRNA can be directly introduced into a cell in a form that is capable of binding to target EphA mRNA transcripts. To increase durability and membrane-permeability the siRNA may be combined or modified with liposomes, poly-L-lysine, lipids, cholesterol, lipofectine or derivatives thereof. In certain aspects cholesterol-conjugated siRNA can be used (see, Song et al., 2003).

2. EphA Protein Targeted Molecules

It is contemplated that nucleic acid aptamers that bind to a EphA protein may be used to reduce expression and/or activity of an EphA protein. In general an aptamer can bind to an Eph protein in a manner similar to an antibody as described above and may, for example, reduce ligand binding, phosphorylation, nuclear translocation or binding partner interaction of a EphA protein.

Methods for selecting aptamers by using recombinant protein, such as EphA5 protein or a domain thereof, to purify nucleic acid aptamers from a library, are well known in the art. The technique known as SELEX and can also be automated to enhance the speed and efficacy of selection, for example see U.S. Pat. Nos. 6,569,620 and 6,716,580. Aptamers identified to bind to an EphA can then be screened for the ability to bind to specific domains of an EphA or to compete with EphA ligands or antibodies (such as 11C12).

Methods for synthesizing and purifying nucleic acids, such as EphA-binding aptamers are well known to those in the art. For example DNA aptamers may be synthesized by PCR, while RNA aptamers can be generated by in vitro transcription. In embodiments, large scale preparation of aptamers may be accomplished by chemical synthesis, this method allows for DNA, RNA and chemically modified oligonucleotides to be incorporated into to the specific aptamer sequence.

### IV. DETECTING EPHA ACTIVITY OR EXPRESSION

Certain embodiments of the invention concern detecting, either in vivo or in a sample, EphA expression or activation. For example, in some embodiments, EphA5 expression can be detected by detecting protein in or on the surface of cells. In some embodiments EphA5 activation is detected, for example by detecting EphA5 phosphorylation, detecting nuclear EphA5, or by detecting EphA5 binding to pATM. Thus, certain methods for detecting EphA5 activation involve detecting EphA5 intracellular localization (or co-localization). In some aspects, a phosphor-specific anti-EphA5 antibody or aptamer can be used to detect EphA5 activation either in vitro or in vivo.

A. Immunodetection Methods

In some embodiments, the present invention concerns immunodetection methods for binding, purifying, removing, quantifying and/or otherwise generally detecting biological components such as EphA protein components. EphA antibodies prepared in accordance with the present invention may be employed to detect EphA expression and/or EphA activation. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing an EphA protein, polypeptide and/or peptide, and contacting the sample with a first anti-EphA antibody in accordance with the present invention, as the case may be, under conditions effective to allow the formation of immunocomplexes.

These methods include methods for purifying wild type and/or mutant EphA proteins, polypeptides and/or peptides as may be employed in purifying wild type and/or mutant EphA proteins, polypeptides and/or peptides from patients' samples and/or for purifying recombinantly expressed wild type or mutant EphA proteins, polypeptides and/or peptides. In these instances, the antibody removes the antigenic wild type and/or mutant EphA protein, polypeptide and/or peptide component from a sample. The antibody will preferably be linked to a solid support, such as in the form of a column matrix, and the sample suspected of containing the wild type or mutant EphA protein antigenic component will be applied to the immobilized antibody. The unwanted components will be washed from the column, leaving the antigen immunocomplexed to the immobilized antibody, which wild type or mutant EphA protein antigen is then collected by removing the wild type or mutant EphA protein and/or peptide from the column.

The immunobinding methods also include methods for detecting and quantifying the amount of a EphA or activated EphA in a sample. Here, one would obtain a sample suspected of containing EphA and contact the sample with an antibody and then detect and quantify the amount of immune complexes formed under the specific conditions.

In terms of antigen detection, the biological sample analyzed may be any sample that is suspected of containing a cell expressing EphA, such as a breast or prostate tissue section or specimen, a homogenized tissue extract, or any biological fluid. Hyperproliferative diseases that may be suspected of expressing EphA or activated EphA, for example can be detected as detailed herein.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any EphA protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

In some embodiments, an EphA antibody employed in the detection may itself be linked to a detectable label, wherein one would then simply detect this label, thereby allowing the amount of the primary immune complexes in the composition to be determined. In some embodiments, the first antibody that becomes bound within the primary immune complexes may be detected by means of a second binding ligand that has binding affinity for the antibody. In some embodiments, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

The immunodetection methods of the present invention have evident utility in the diagnosis and prognosis of conditions such as various forms of hyperproliferative diseases, such as cancer, including leukemia, for example. Here, a biological and/or clinical sample suspected of containing a wild type or mutant EphA protein, polypeptide, peptide and/or mutant is used. However, these embodiments also have applications to non-clinical samples, such as in the titering of antigen or antibody samples, for example in the selection of hybridomas.

In the clinical diagnosis and/or monitoring of patients with various forms of hyperproliferative disease, such as cancer, for example, leukemia, the detection of EphA mutant, and/or an alteration in the expression or activation of EphA, in comparison to the levels in a corresponding biological sample from a normal subject is indicative of a patient with hyperproliferative disease, such as cancer. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive.

### ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In some embodiments, the anti-EphA antibodies of the invention are immobilized onto a selected surface exhibiting protein affinity, such as a well in a polystyrene microtiter plate. Then, a test composition suspected of containing the wild type and/or mutant EphA protein antigen, such as a clinical sample, is added to the wells. After binding and/or washing to remove non-specifically bound immune complexes, the bound wild type and/or mutant EphA protein antigen may be detected. Detection is generally achieved by the addition of another anti EphA antibody that is linked to a detectable label. This type of ELISA is a simple “sandwich ELISA”. Detection may also be achieved by the addition of a second anti-EphA antibody, followed by the addition of a third antibody that has binding affinity for the second antibody, with the third antibody being linked to a detectable label.

In some embodiments, the samples suspected of containing the wild type and/or mutant EphA protein antigen are immobilized onto the well surface and/or then contacted with the anti-EphA antibodies of the invention. After binding and/or washing to remove non-specifically bound immune complexes, the bound anti-EphA antibodies are detected. Where the initial anti-EphA antibodies are linked to a detectable label, the immune complexes may be detected directly. Again, the immune complexes may be detected using a second antibody that has binding affinity for the first anti-EphA antibody, with the second antibody being linked to a detectable label.

In some embodiments, the wild type and/or mutant EphA proteins, polypeptides and/or peptides are immobilized. In some embodiments, ELISA involves the use of antibody competition in the detection. In this ELISA, labeled antibodies against wild type or mutant EphA protein are added to the wells, allowed to bind, and/or detected by means of their label. The amount of wild type or mutant EphA protein antigen in an unknown sample is then determined by mixing the sample with the labeled antibodies against wild type and/or mutant EphA before and/or during incubation with coated wells. The presence of wild type and/or mutant EphA protein in the sample acts to reduce the amount of antibody against wild type or mutant protein available for binding to the well and thus reduces the ultimate signal. This is also appropriate for detecting antibodies against wild type or mutant EphA protein in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In some embodiments, a secondary or tertiary detection means is used rather than a direct procedure. In some such embodiments, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody may have an associated label to allow detection. In some embodiments, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween). After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H2O2, in the case of peroxidase as the enzyme label. Quantification is then achieved by measuring the degree of color generated, e.g., using a visible spectra spectrophotometer.

1. Immunohistochemistry

Antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in 70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Immunoelectron Microscopy

Antibodies of the present invention may also be used in conjunction with electron microscopy to identify intracellular tissue components. Briefly, an electron-dense label is conjugated directly or indirectly to an anti-EphA antibody. Examples of electron-dense labels according to the invention are ferritin and gold. The electron-dense label absorbs electrons and can be visualized by the electron microscope.

2. Immunodetection Kits

In some embodiments, the present invention concerns immunodetection kits for use with the immunodetection methods described above. As EphA antibodies are generally used to detect wild type and/or mutant EphA proteins, polypeptides and/or peptides, the antibodies will preferably be included in the kit. However, kits including both such components may be provided Immunodetection kits will thus comprise, in suitable container means, a first antibody that binds to a wild type and/or mutant EphA protein, polypeptide and/or peptide, and/or optionally, an immunodetection reagent and/or further optionally, a wild type and/or mutant EphA protein, polypeptide and/or peptide.

In some embodiments, monoclonal antibodies will be used. In certain embodiments, the first antibody that binds to the wild type and/or mutant EphA protein, polypeptide and/or peptide may be pre-bound to a solid support, such as a column matrix and/or well of a microtitre plate.

Immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be employed in connection with the present invention.

Kits in accordance with the present invention may further comprise a suitably aliquoted composition of the wild type and/or mutant EphA protein, polypeptide and/or polypeptide, whether labeled and/or unlabeled, as may be used to prepare a standard curve for a detection assay. Provided kits may contain antibody-label conjugates either in fully conjugated form, in the form of intermediates, and/or as separate moieties to be conjugated by the user of the kit. The components of the kits may be packaged either in aqueous media and/or in lyophilized form.

The container means of the kits will generally include at least one vial, test tube, flask, bottle, syringe and/or other container means, into which the antibody may be placed, and/or preferably, suitably aliquoted. Where wild type and/or mutant EphA protein, polypeptide and/or peptide, and/or a second and/or third binding ligand and/or additional component is provided, the kit will also generally contain a second, third and/or other additional container into which this ligand and/or component may be placed. The kits of the present invention will also typically include a means for containing the antibody, antigen, and/or any other reagent containers in close confinement for commercial sale. Such containers may include injection and/or blow-molded plastic containers into which the desired vials are retained.

B. Nucleic Acid Detection

In some embodiments, assessing expression of an EphA may involve quantitating mRNA. Northern blotting techniques are well known to those of skill in the art. Northern blotting involves the use of RNA as a target. Briefly, a probe is used to target an RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (such as a labeled probe) under conditions that promote denaturation and rehybridization. Because the probe is designed to base pair with the target, the probe will binding a portion of the target sequence under renaturing conditions. Unbound probe is then removed, and detection is accomplished.

In some embodiments, nucleic acids are quantitated following gel separation and staining with ethidium bromide and visualization under UV light. In some embodiments, if the nucleic acid results from a synthesis or amplification using integral radio- or fluorometrically-labeled nucleotides, the products can then be exposed to x-ray film or visualized under the appropriate stimulating spectra, following separation.

In some embodiments, visualization is achieved indirectly. Following separation of nucleic acids, a labeled nucleic acid is brought into contact with the target sequence. The probe is conjugated to a chromophore or a radiolabel. In another embodiment, the probe is conjugated to a binding partner, such as an antibody or biotin, and the other member of the binding pair carries a detectable moiety. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In some embodiments, the amplification products described above may be subjected to sequence analysis to identify specific kinds of variations using standard sequence analysis techniques. Within certain methods, exhaustive analysis of genes is carried out by sequence analysis using primer sets designed for optimal sequencing. The present embodiments provide methods by which any or all of these types of analyses may be used. Using the sequences disclosed herein, oligonucleotide primers may be designed to permit the amplification of sequences throughout a EphA gene (or protein coding sequence) that may then be analyzed by direct sequencing.

In some embodiments, reverse transcription (RT) of RNA to cDNA followed by relative quantitative PCR™ (RT-PCR™) can be used to determine the relative concentrations of specific mRNA species isolated from patients (e.g., an EphA coding RNA). By determining that the concentration of a specific mRNA species varies, it is shown that the gene encoding the specific mRNA species is differentially expressed.

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ experiment to successfully determine the relative abundances of a particular mRNA species is that relative concentrations of the amplifiable cDNAs must be normalized to some independent standard. The goal of an RT-PCR™ experiment is to determine the abundance of a particular mRNA species relative to the average abundance of all mRNA species in the sample.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

### V. COMBINATION THERAPIES

In order to increase the effectiveness of EphA-targeted therapies of the embodiments, it may be desirable to combine these compositions with other agents effective in the treatment of the disease of interest. For example, in certain aspects an EphA-targeted therapy is administered in conjunction with a DNA damaging agent (e.g., radiation or chemotherapeutic agents, among others).

As a non-limiting example, the treatment of a cell hyperproliferative disorder, such as cancer, may be implemented with therapeutics of the present invention along with a DNA damaging agent. As used herein a “DNA damaging agent” should be understood as a reference to any treatment (e.g., radiation exposure) or molecules (e.g., a chemotherapeutic) that acts to damage cellular DNA. Examples of DNA damaging agents include, but are not limited to, the traditionally understood chemotherapy agents such as actinomycin D, arsenic trioxide, asparaginase, bleomycin, busulfan, carboplatin, carmustine, chlorambucil, cisplatin, corticosteroids, cyclophosphamide, daunorubicin, docetaxel, doxorubicin, epirubicin, etoposide, fludarabine, fluorouracil, gemcitabine, hydroxyurea, idarubicin, ifosfamide, irinotecan, lomustine, melphalan, mercaptopurine, methotrexate, mitomycin, mitoxantrone, oxaliplatin, paclitaxel, procarbizine, raltitrexed, streptozocin, thioguanine, thiotepa, topotecan, treosulfan, vinblastine, vincristine, vindesine, vinorelbine. Other means of inducing DNA damage include ionizing radiation as well as the use of molecules such as inhibitors of poly-(ADP ribosyl) transferase (PARP) or agents which induce DNA damage as part of a synergistic process with another agent, for example e.g., gemcitabine or irinotecan and CHK1/2 inhibitors such as CBP-501 or AZD7762.

Treatment with an EphA-targeted therapy may precede or follow a DNA damaging agent treatment (or other anti-cancer agent treatment) by intervals ranging from minutes to weeks. In embodiments where the EphA-targeted therapy and DNA damaging agent (or other anti-cancer agent treatment) are applied separately to a cell or patient, one would generally ensure that a significant period of time did not expire between the time of each delivery, such that the DNA damaging agent and the EphA-targeted therapy would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly where several days (e.g., 2, 3, 4, 5, 6 or 7 days) to several weeks (e.g., 1, 2, 3, 4, 5, 6, 7 or 8 weeks) lapse between the respective administrations.

Various combinations may be employed, where the EphA5-targeted therapy is “A” and the secondary agent, such as a DNA damaging agent (e.g., a radiotherapy or chemotherapy), is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of the EphA5-targeted therapy of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

In some embodiments an EphA-targeted therapy can be administered in conjunction with DNA damaging agent and/or another anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with a EphA-targeting agent and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the a EphA-targeting agent and the other includes the second agent(s). In some embodiments, an EphA-targeting agent can be one agent, and a DNA damaging agent can be the other agent.

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, many chemotherapy agents are DNA damaging agents as outlined above. An agent may, for example, be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include without limitation alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum, and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

In some embodiments, a DNA damaging agnet for use according to the embodiments can be a radiation therapy. For example, a radiation therapy can be administered by exposing a patient (or a part of the patient) to γ-rays, X-rays, and/or by the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic composition and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

In some embodiments, an EphA-targeted therapy can be administered in conjunction with an immunotherapy Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In some embodiments, an immunotherapy can comprise administering one or more tumor antigens to a patient (e.g., a cancer vaccine) or exposing a patient's immune cells to such a cancer antigen (e.g., an autologous cellular immunotherapy). Antigens that can be used in such therapeutic approaches include, but are not limited to, prostatic acid phosphotase (PAP), MUC1, HER2/neu, 5T4 and whole killed cancer cells.

Immunotherapy, thus, could be used as part of a combined therapy, in conjunction with a EphA5-targeted therapy of the present invention. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

In some embodiments, an EphA-targeted therapy can be administered in conjunction with a gene therapy (e.g., a therapeutic polynucleotide composition). Viral vectors for the expression of a gene product are well known in the art, and include such eukaryotic expression systems as adenoviruses, adeno-associated viruses, retroviruses, herpesviruses, lentiviruses, poxviruses including vaccinia viruses, and papilloma viruses, including SV40. Alternatively, the administration of expression constructs can be accomplished with lipid based vectors such as liposomes or DOTAP:cholesterol vesicles. All of these method are well known in the art (see, e.g. Sambrook et al., 1989; Ausubel et al., 1998; Ausubel, 1996).

Delivery of a vector encoding one of the following gene products will have a combined anti-hyperproliferative effect on target tissues.

In some embodiments, an EphA-targeted therapy can be administered in conjunction with a surgical therapy (e.g., before after or during surgical resection of a tumor). Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some embodiments, EphA-targeted therapies are administered in conjunction with an anti-inflammatory agent. An anti-inflammatory agent is defined herein to refer to an agent that is known or suspected to be of benefit in the treatment or prevention of inflammation in a subject. Corticosteroids are a major class of anti-inflammatory agent. The corticosteroids may be short, medium, or long acting, and may be delivered in a variety of methods. A non-limiting list of corticosteroids contemplated in the present invention include the oral corticosteroids such as: cortisone, hydrocortisone, prednisone, and dexamethasone.

Another major class of anti-inflammatory agents are non-steroidal anti-inflammatory agents. Non-steroidal anti-inflammatory agents include a class of drugs used in the treatment of inflammation and pain. The exact mode of action of this class of drugs is unknown. Examples of members of this class of agents include, but are not limited to, ibuprofen, ketoprofen, flurbiprofen, nabumetone, piroxicam, naproxen, diclofenac, indomethacin, sulindac, tolmetin, etodolac, flufenamic acid, diflunisal, oxaprozin, rofecoxib, and celecoxib. One of ordinary skill in the art would be familiar with these agents. Included in this category are salicylates and derivates of salicylates, such as acetyl salicylic acid, sodium salicylate, choline salicylate, choline magnesium salicylate and diflunisal.

Other anti-inflammatory agents include anti-rheumatic agents, such as gold salts (e.g., gold sodium thiomalate, aurothioglucose, and auranofin), anti-rheumatic agents (e.g., chloroquine, hydroxychloroquine, and penicillamine), antihistamines (e.g., diphenhydramine, chlorpheniramine, clemastine, hydroxyzine, and triprolidine), and immunosuppressive agents (e.g., methotrexate, mechlorethamine, cyclophosphamide, chlorambucil, cyclosporine, and azathioprine). Other immunosuppressive agent contemplated by the present invention is tacrolimus and everolimus. Tacrolimus suppresses interleukin-2 production associated with T-cell activation, inhibits differentiation and proliferation of cytotoxic T cells. Today, it is recognized worldwide as the cornerstone of immunosuppressant therapy. One of ordinary skill in the art would be familiar with these agents, and other members of this class of agents, as well as the mechanism of actions of these agents and indications for use of these agents.

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### VI. ADMINISTRATION

An EphA-targeted therapy of the embodiments can be administered in a number of ways depending upon whether local or systemic treatment is desired, and upon the area to be treated. In some embodiments, an EphA-targeted therapy is administered in a formulation comprising a DNA damaging agent as detailed above.

Administration may be topical (including ophthalmic, vaginal, rectal, intranasal, transdermal), oral, nasal, bronchial or parenteral, for example, by intravenous drip, subcutaneous, intraperitoneal or intramuscular injection. In the case a patient having a tumor a composition can be administered proximal to the site of a tumor, such as by an intratumoral injection. Administration can be either rapid as by injection or over a period of time as by slow infusion or administration of slow release formulations.

In some embodiments EphA-targeting agents may be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. For example, cationic lipids may be included in the formulation to facilitate uptake of the agents.

The actual dosage amount of a composition of the present invention administered to a subject can be determined by physical and physiological factors such as body weight, severity of condition, the type of disease being treated, previous or concurrent therapeutic interventions (e.g., use of DNA damaging agents), idiopathy of the patient and on the route of administration. The practitioner responsible for administration will, in any event, determine the concentration of active ingredient(s) in a composition and appropriate dose(s) for the individual subject.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired. In some embodiments, dosages can be determined by measuring for example changes in Eph activation in a subject. For example, an effective amount of an EphA-targeting agent can be an amount that reduces EphA (e.g., EphA5) phosphorylation by at least about 10%, 20%, 30%, 40%, 50%, 60%, 70%, 80%, 90%, 95%, 98% or more.

Precise amounts of the therapeutic composition may also depend on the judgment of the practitioner and are peculiar to each individual. Factors affecting the dose include the physical and clinical state of the patient, the route of administration, the intended goal of treatment (e.g., alleviation of symptoms versus attaining a particular serum insulin or glucose concentration) and the potency, stability and toxicity of the particular therapeutic substance.

In some embodiments, pharmaceutical compositions may comprise, for example, at least about 0.1% of an active agent, such as an EphA-targeting agent and/or a DNA damaging agent. In some embodiments, the an active agent may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein of an active agent. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The following examples are intended to illustrate but not limit the present invention.

### EXAMPLES

### Example 1

### EphA5 Expression Affects Cell Proliferation

Two independent lentiviral vectors expressing anti-EphA5 small hairpin RNAs (shRNA) or a negative control scrambled shRNA were used to evaluate the biological activity of EphA5 in human lung cancer. The silencing experiments were performed with retroviral vectors pLKO.1 from the TRC lentiviral shRNA library (Open Biosystems) expressing specific shRNAs for human EPHA5 (oligonucleotide TRCN0000006413, referred to as EphA5-shRNA 1, and TRCN0000006415, referred to as EphA5-shRNA 2).

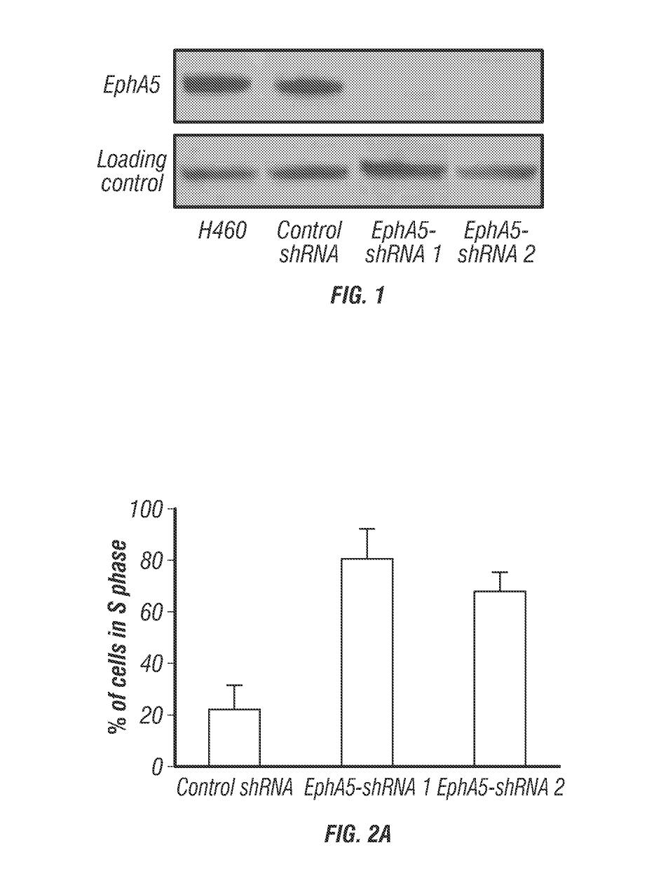

Western blot analysis confirmed marked reduction of EphA5 in lung cancer cells infected with the lentiviruses expressing anti-EphA5 shRNAs while those cells infected with the control shRNA showed no alterations in EphA5 expression (FIG. 1).

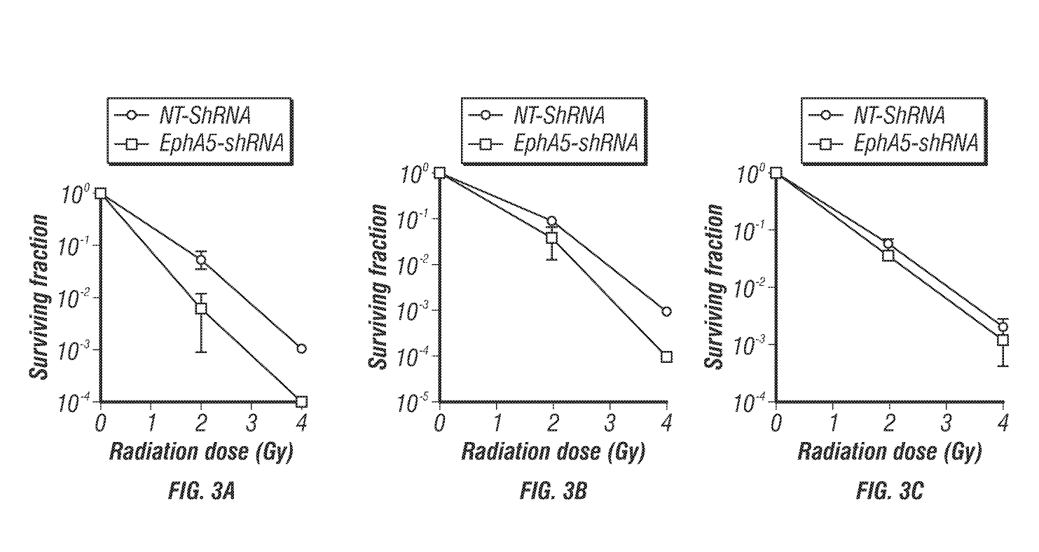

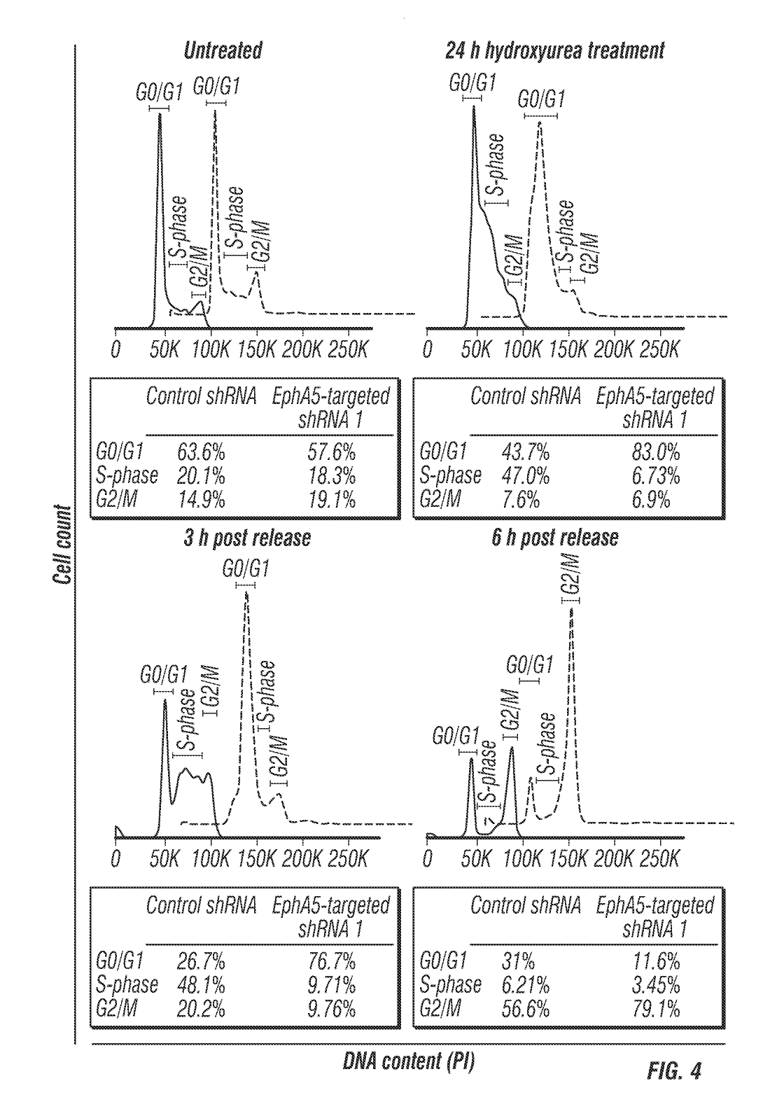

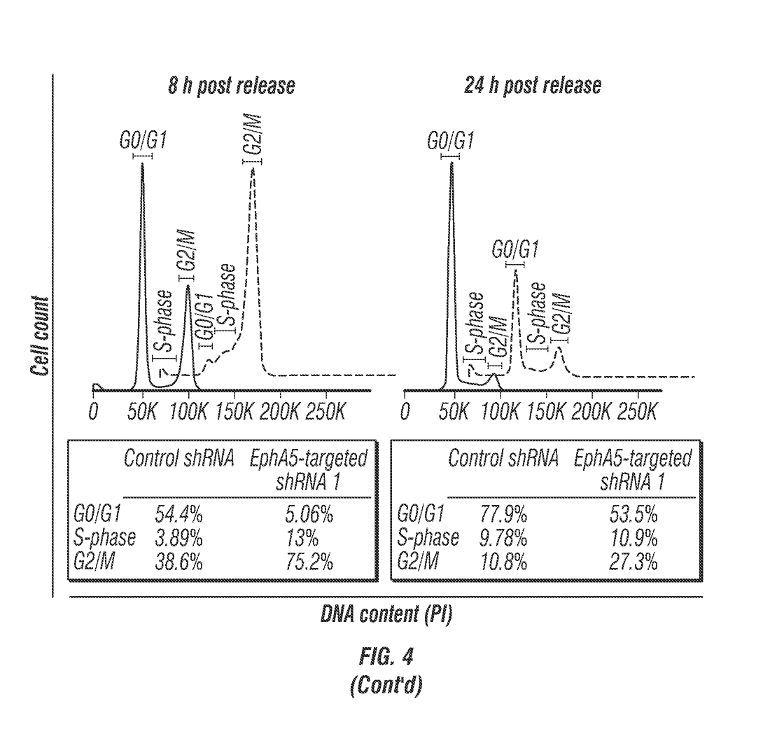

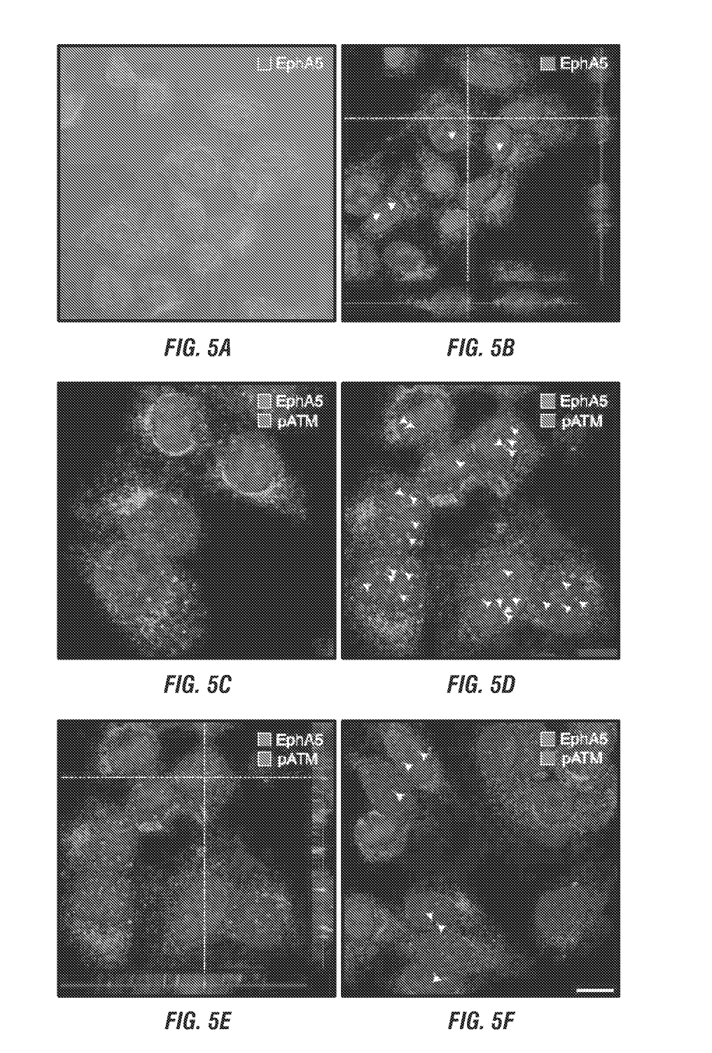

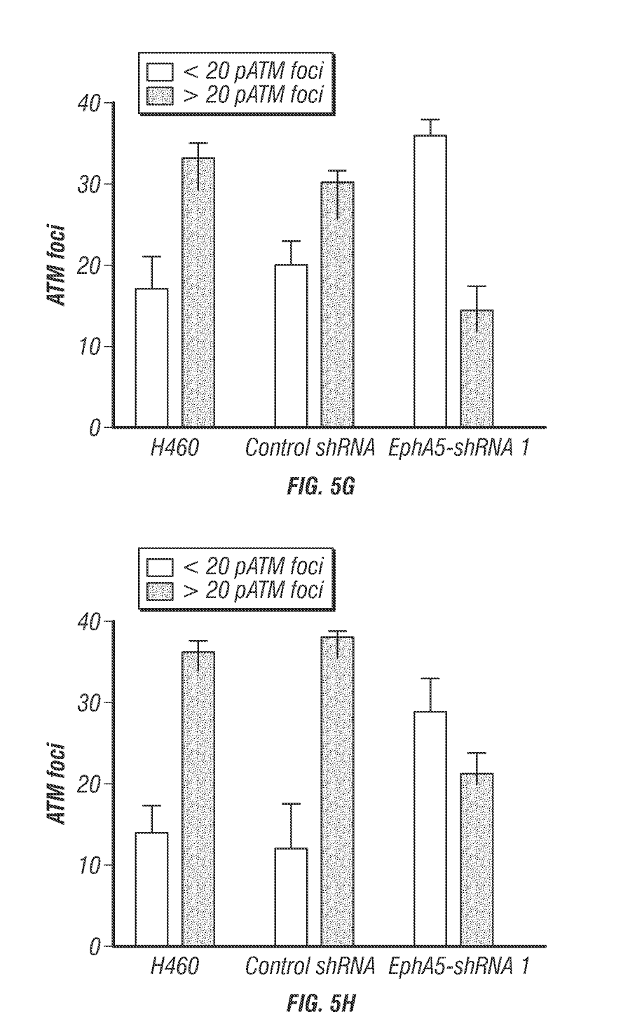

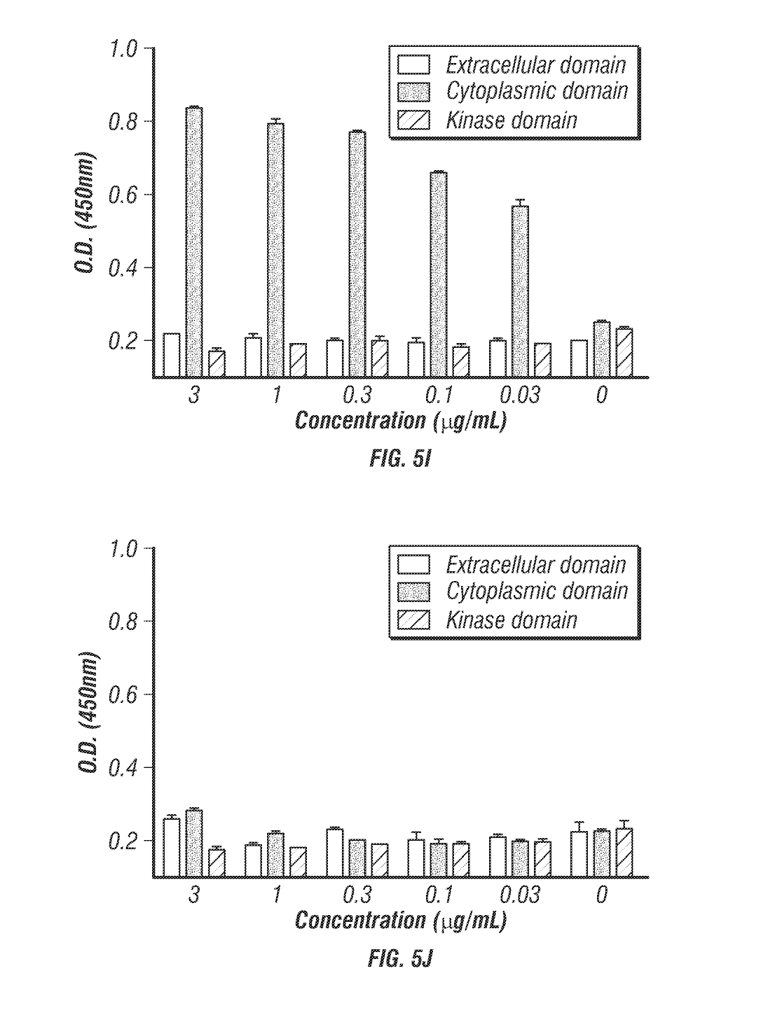

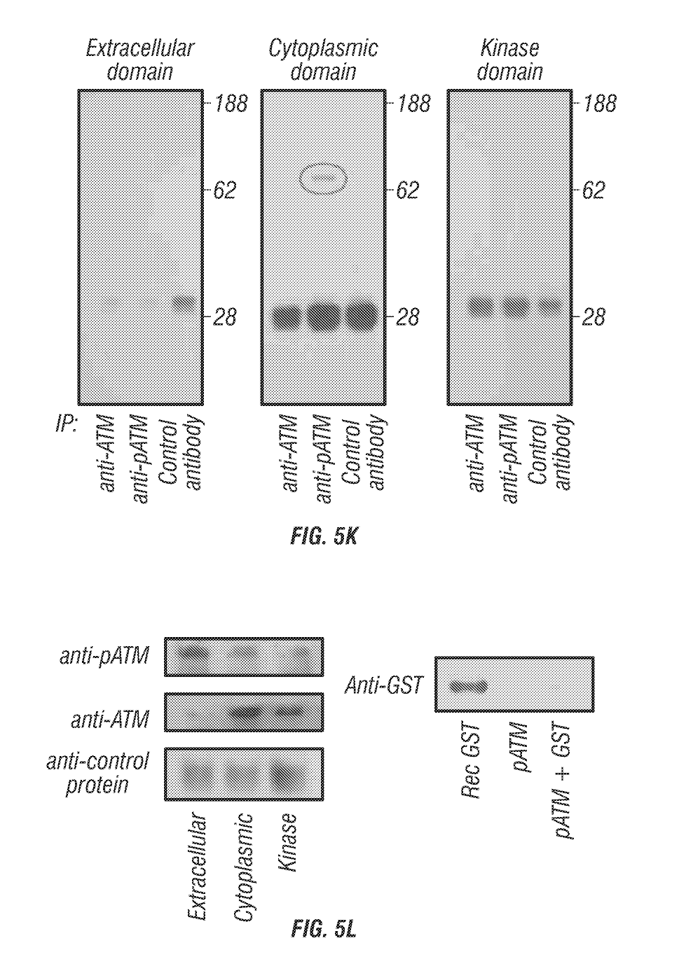

An analysis of DNA content and bromodeoxyuridine (BrdU) incorporation in the matched cell lines was used to evaluate the role of EphA5 in lung cancer cells proliferation (FIG. 2). Depletion of EphA5 expression was sufficient to cause cell cycle arrest in S phase (FIG. 2A). A functional evaluation of cell cycle checkpoint activity after ionizing radiation (IR)-induced DNA damage was the performed in G0/G1-synchronized cells (FIG. 2, B to E). In comparison to non-irradiated cells (FIG. 2B), increasing doses of radiation elicited different cell cycle effects that were dependent on EphA5 expression. After IR with 3 Gy (FIG. 2C) and 5 Gy (FIG. 2D), EphA5-positive cells were arrested in G1, a result confirming the capacity of these cells to sense DNA damage and to initiate the DNA-damage repair machinery. In contrast, EphA5-negative cells progressed through G1/S, a result indicative of critical defects in the assessment of DNA damage caused by IR. Both EphA5-positive and EphA5-negative cells presented comparable responses following 10 Gy IR (FIG. 2E), an indication of a functional G2/M checkpoint. Compared to their respective controls, H1299 and H522 EphA5-negative cells showed a marked reduction in survival upon IR-induced DNA damage, again supporting a role for EphA5 in DNA damage response (FIG. 3). Interruption of DNA synthesis promoted by treatment of tumor cells with hydroxyurea produced similar results (FIG. 4). Hydroxyurea-treated EphA5-positive cells were arrested in G1 after cell cycle release into high-serum media, whereas EphA5-negative cells were not (FIG. 5). Altogether, it was reasoned that these findings indicate that DNA damage-induced biochemical events triggering G1/S checkpoint in EphA5-negative cells might be defective.

To further evaluate the role of EphA5 in DNA damage repair and genomic stability, clonogenic survival following IR was assessed for EphA5-positive and EphA5-negative lung cancer cells. Compared to the EphA5-expressing cells, EphA5-depleted cells showed a marked reduction in clonogenic survival (FIG. 2F). Moreover, ectopic expression of EphA5 in normal human pulmonary fibroblasts (HPF) induced increased radioresistance (FIG. 2G). Together, these results establish EphA5 overexpression is one mechanism of cellular radioresistance, with potential implications for the therapeutic use of IR in lung cancer.

### Example 2

### Mechanism of EphA5 in Human Lung Cancer IR Resistance

Given the role of EphA5 in cell cycle control following IR and in intrinsic cellular radiosensitivity, studies were performed to determine whether EphA5 translocates to the nucleus of irradiated cells. Confocal microscopy was used to study the localization and distribution of EphA5 in lung cancer cells. Cytoplasmic and perinuclear staining were readily detectable in control (non-irradiated) human lung cancer cells immunostained with two distinct anti-EphA5 antibodies (FIGS. 5, A and B). Small amounts of EphA5 were also observed inside the nucleus of cells (FIG. 5B, indicated by arrowheads). Next, irradiated tumor cells were co-immunostained with anti-phosphorylated ATM (pATM) and anti-EphA5 antibodies, and studied the distribution of EphA5 and the formation of ATM foci in sites of DNA damage. Notably, prior to irradiation, co-localization of pATM and EphA5 was not detectable (FIG. 5C). However, co-localization of these proteins was observed in ˜30% of ATM foci detected after 10 min of IR (FIGS. 5, D and E, arrowheads). Six hours later, co-localized EphA5/pATM nuclear foci were detectable, although in small numbers (FIG. 5F). To investigate whether EphA5 influences ATM activity at sites of DNA damage, next pATM focus formation in lung cancer cells was analyzed as a function of EphA5 expression. Quantitative analysis demonstrated a marked reduction in the number of nuclear pATM foci in EphA5-negative cells after IR (FIGS. 5, G and H). This result indicates that EphA5 may be essential for recruiting, activating, and/or stabilizing ATM at sites of DNA injury.

To further evaluate the molecular interaction between ATM and EphA5, immunocapture assays were used to detect the binding of ATM or pATM to domains of EphA5. Three constructs comprising the EphA5 extracellular domain (residues 25-573), the complete cytoplasmic domain (residues 595-1037), and the kinase domain (residues 655-956) were exposed to either pATM (FIG. 5I) or ATM (FIG. 5J) immobilized on 96-well plates. A clear protein-protein interaction between pATM and the cytoplasmic domain of EphA5 was observed (FIG. 5I). These results were confirmed in a second independent binding assay, in which ATM and pATM were immobilized on magnetic beads and exposed to each of the EphA5 constructs. Again, only the complete EphA5 cytoplasmic domain showed direct physical interaction with pATM (FIG. 5K, indicated by the circle). Collectively, these results clearly show that EphA5 and ATM are molecular binding partners. This protein-protein interaction predominantly observed at sites of DNA injury, and indicates that EphA5 is an important regulator of DNA repair in human lung cancer.

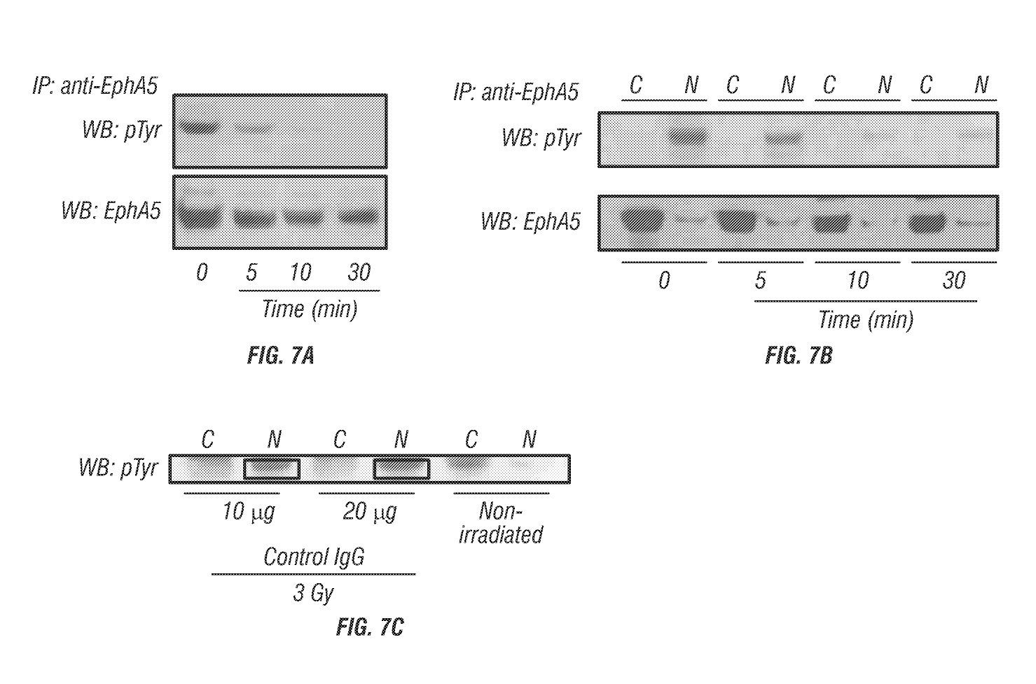

The phosphorylation status of EphA5 in cell nuclei was next investigated. Nuclear extracts of irradiated and non-irradiated cells were subjected to immunoprecipitation with anti-EphA5 antibody followed by immunoblotting with either anti-phosphotyrosine antibody (pTyr) or with a specific anti-EphA5 antibody (FIG. 6A). Nuclear levels of EphA5 varied post irradiation; namely, phosphorylated EphA5 was undetectable prior to IR and it was found only in the nucleus of irradiated tumor cells (FIG. 7A). To determine whether nuclear EphA5 import also occurs after ligand-dependent EphA5 activation, human lung cancer cells were treated with an endogenous EphA5 ligand (FIG. 7A) and subjected cytoplasmic and nuclear fractions to the same procedure described above (FIG. 7B). Similarly, phosphorylated EphA5 was found only in the nuclei of activated cells (FIG. 7B). Collectively, these data confirm the nuclear translocation of EphA5 upon exogenous stimulation, and suggest that translocation may be dependent on EphA5 phosphorylation.

### Example 3

### Anti-EphA5 Antibody Sensitizes Human Lung Cancer Cells to IR

Given the potential broad relevance of EphA5 in human lung cancer, monoclonal antibodies against EphA5 were produced and tested for their ability to inhibit the activity of the receptor in vitro. Hybridomas were prepared with splenocytes from a mouse immunized with the recombinant extracellular domain of EphA5 and screened by flow cytometry and immunoprecipitation. By these criteria, a monoclonal antibody against EphA5 (termed 11C12) was selected for functional studies.

To characterize the effect of 11C12 on the response of cells to IR, human lung cancer cells were exposed to increasing doses of IR in the presence or absence of 11C12 or control isotype IgG, and assayed for clonogenicity 10 days after irradiation (FIGS. 6, B and C). A marked reduction in clonogenicity was observed after cells were exposed to different concentrations of 11C12 (FIGS. 6, B and D), but not to control IgG. Similar results were observed with a second human cancer cell line (H522), thus confirming the capacity of 11C12 to radiosensitize lung tumor cells (FIG. 6C). Because phosphorylation of EphA5 seemed to be functionally related to its presence in cell nuclei, it was tested whether 11C12 would sensitize cells via inhibition of receptor phosphorylation and nuclear activity. Indeed, phosphorylated EphA5 was not detected in the nucleus of irradiated cells previously treated with increasing doses of 11C12 (FIG. 6E); isotype IgG served as the negative control (FIG. 7C).

To assess the potential clinical relevance of these findings, immunohistochemical staining was performed with a commercial antibody against EphA5 on a lung cancer tissue microarray (TMA) comprising a cohort of tumor specimens (n=255) obtained from patients diagnosed with non-small lung carcinoma (NSCLC), including adenocarcinomas (ACC) and squamous cell carcinomas (SCC), of differing clinical stages (Prudkin et al., 2008). This large panel of patient-derived samples has well-annotated clinical-pathologic features that include—but are not limited to—smoking history, recurrence, and survival data (Prudkin et al., 2008). The presence of EphA5 in these sets of samples was assessed according to levels and scores of expression, and data were quantified independently by two expert lung cancer pathologists. Expression was detected predominantly in the cytoplasm and membrane of ˜70% of analyzed NSCLC (178 out of 255) samples (Table 1). EphA5 was found to be widely expressed in human lung cancer, and the frequency of positive cases was significantly higher in SCC (81%) compared to ACC (62%) (Table 1 and FIG. 8A) (Wilcoxon rank sum test, P<0.0001). This result is particularly tantalizing given the greater unmet needs in the therapy of human squamous cell carcinoma of the lung.

TABLE 1
Relationship between EphA5 expression and clinicopathologic
characteristics in 255 NSCLC patients.

| | | EphA5 | | |
|---|---|---|---|---|
| | | Negative | Positive | |
| Characteristics | | n (%) | n (%) | P-value |
| Histology | ACC | 58 (38.2%) | 94 (61.8%) | 0.0024 |
| | SCC | 18 (19.1%) | 76 (80.9%) | |
| | Other | 1 (11.1%) | 8 (88.9%) | |
| Gender | Female | 28 (23%) | 94 (77%) | 0.0158 |
| | Male | 49 (36.8%) | 84 (63.2%) | |
| Race | Black | 7 (41.2%) | 10 (58.8%) | 0.5203 |
| | Hispanic | 3 (42.9%) | 4 (57.1%) | |
| | Asian | 1 (33.3%) | 2 (66.7%) | |
| | White | 66 (28.9%) | 162 (71.1%) | |
| Smoker | Never | 5 (22.7%) | 17 (77.3%) | 0.1470 |
| | Former | 30 (25.4%) | 88 (74.6%) | |
| | Current | 42 (36.5%) | 73 (63.5%) | |
| Pathstage | IA | 14 (23%) | 47 (77%) | 0.5776 |
| | IB | 29 (29.9%) | 68 (70.1%) | |
| | IIA | 5 (38.5%) | 8 (61.5%) | |
| | IIB | 13 (34.2%) | 25 (65.8%) | |
| | IIIA | 16 (34.8%) | 30 (65.2%) | |
| Stage | I | 43 (27.2%) | 115 (72.8%) | 0.4160 |
| | II | 18 (35.3%) | 33 (64.7%) | |
| | IIIA | 16 (34.8%) | 30 (65.2%) | |
| Differentiation | Poorly | 31 (36%) | 55 (64%) | 0.1176 |
| | Moderate | 37 (25.5%) | 108 (74.5%) | |
| | Well | 7 (34.8%) | 9 (56.3%) | |

Because these findings pointed to a direct effect of EphA5 in the resistance to radiotherapy, EphA5 expression was next examined in an independent cohort of surgically resected tumors from lung cancer patients (n=23) with stage III NSCLC that had all undergone radiotherapy following “margin-negative” surgery. The cohort included patients with biopsy-confirmed local failure within the radiation field (n=12) and with no evidence of local failure (n=11); histologies included ACC (74%) and SCC (26%), similarly distributed between the two groups. Consistent with the mechanistic hypothesis, high levels of EphA5 were clearly correlated with a lack of response to radiotherapy (FIGS. 8, C and D). More importantly, there was a direct correlation between EphA5 expression and mortality (FIG. 8E).

Thus, these studies establish EphA5 as a novel regulator of DNA damage repair, with direct clinical implications for the response of lung cancer patients to radiation therapy. It was also shown that EphA5 is widely expressed in human lung cancer, and that those patients expressing EphA5 as a molecular marker are at much higher risk for radiotherapy failure and death. Finally, a new monoclonal antibody against EphA5 was produced and demonstrated to sensitize lung cancer cells to radiotherapy. The therapeutic implications of EphA5-targeted therapy, together with its description as a biomarker of radiotherapy failure and poor overall survival, identifies this tyrosine kinase receptor as a critical new target for the establishment of therapeutic strategies for the treatment of lung cancer.

### Example 4

### Characterizing the 11C12 Monoclonal Anti EphA5 Antibody

Nucleic acids encoding 11C12, a murine IgG1 monoclonal anti-EphA5 antibody, were sequenced to determine the sequences of the variable regions (V_{H} and V_{L}). The of the 11C12 V_{H} is provided as SEQ ID NO:1 (nucleic acid) and SEQ ID NO:11 (amino acid) and the sequence for the V_{L} is SEQ ID NO:2 (nucleic acid) and SEQ ID NO:12 (amino acid).

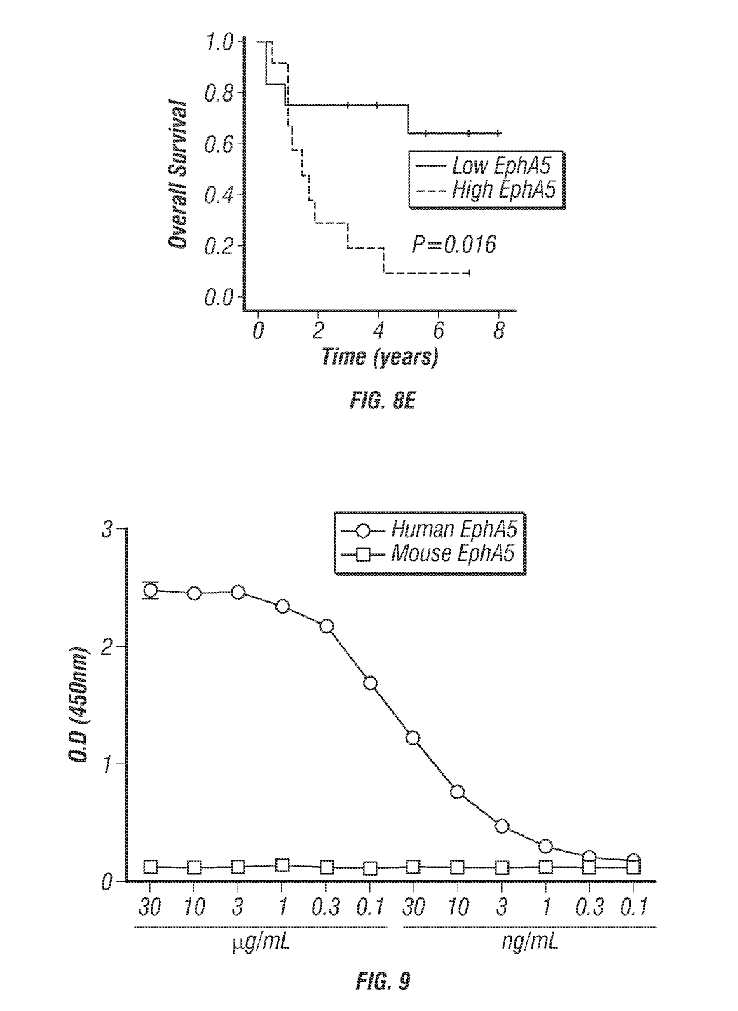

The 11C12 antibody was further characterized by examining its binding characteristics. The antibody was tested in a ELISA assay with decreasing amounts of target human or mouse EphA5 protein. The results of the studies are shown in FIG. 9 and demonstrated 11C12 binding is specific for human EphA5 protein. No binding to murine EphA5 protein was detected. On the other hand, binding of 11C12 to human EphA5 protein, as measured by optical density (OD), decreases as the amount of human EphA5 protein decreases.

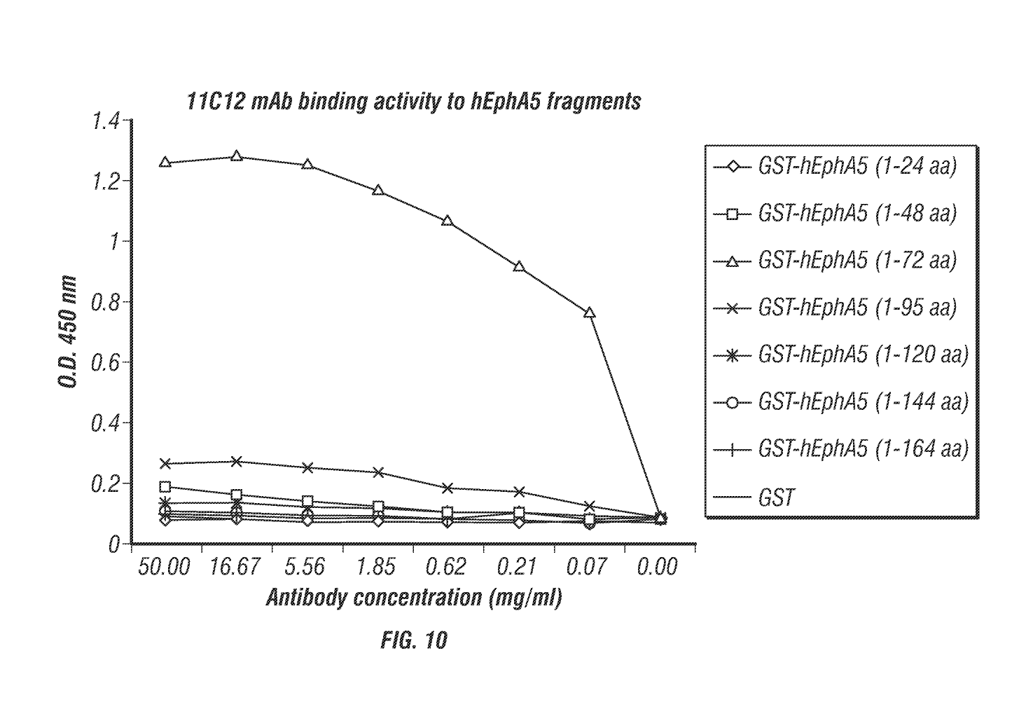

Epitope mapping was performed with protein fragments of human EphA5. The results of these studies demonstrated that the 11C12 monoclonal antibody recognizes a protein sequence between amino acids 304-467 of the human protein. This region is provided as SEQ ID NO:3. Further epitope mapping was performed by ELISA using fusion proteins including EphA5 sequences within the 304-467 region (FIG. 10). The greatest binding was observed with GST-hEphA5 (1-72 aa) indicating that the primary 11C12 binding eptiope is in the first 72 amino acids of the 304-467 region of the hEphA5 protein, this sequence is provided as SEQ ID NO:6.

### Example 5

### Anti-EphA5 Antibodies Reduce Growth of Cancer Cells in Culture

To study the effects of anti-EphA5 antibodies on cell proliferation colony formation assays were performed in the presence of the 11C12 monoclonal antibody. Briefly, H460 cells were seeded in the presence of increasing concentrations of 11C12. The number of cell colonies formed were counted 10 days after the initial plating. Results from these experiments are shown in FIG. 10A and demonstrate that, at the highest concentrations (50 μg and 100 μg per well) a reduction of 30% in the total number of colonies was observed.

To assess the effect of 11C12 on cell proliferation H460 cells were grown in the presence of 100 μg/well of 11C12 or control IgG and cell proliferation was measured in real time by measuring electrical resistance across micro electrodes in the culture dish (using an xCELLigence System assay). Results from these studies are shown in FIG. 10B and demonstrate that cell proliferation is reduced by approximately 30% in the presence of 11C12.

The effects of 11C12 on cell adhesion were also tested using the xCELLigence System. In this case, cells were plated in the presence of 11C12 or a control IgG (each at 100 μg/well). Adhesion of the cells to the culture plate was assessed in real time during the first 6 hours of the experiment. Results show in FIG. 11C demonstrate that 11C12 mediates a small decrease in cell adhesion. Thus, even absent DNA damaging agents, anti-EphA5 antibodies were found to reduce cancer cell proliferation indicating that such antibodies may be used as effective cancer therapeutics.

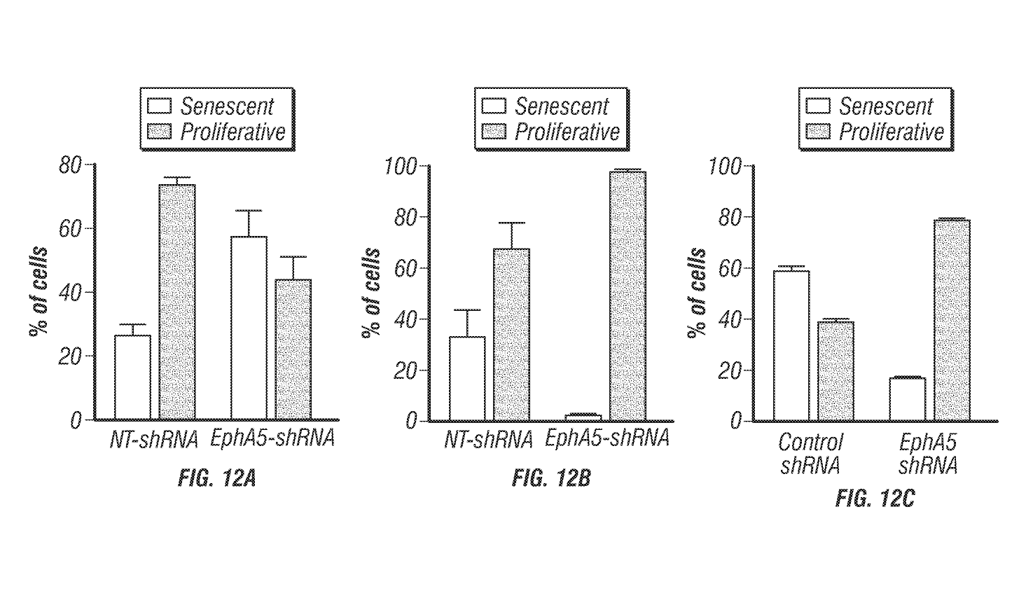

Wild type p53 H460 cells (FIG. 12A), H1299 p53 null cells (FIG. 12B) and H522 (p53 mutated) (FIG. 12C) were used to demonstrate that EphA5-induced senescence depends on the presence and/or activity of p53.

### Example 6

### In Vivo Administration of an Anti-EphA5 Antibody

To determine if cancer cells could be effectively targeted with an anti-EphA5 antibody in vivo nude mice were subcutaneously inoculated with human H460 lung cancer cells. Two weeks post inoculation the tumor bearing mice were administered 3 μg of fluorophore-labeled (IRDye®-labeled) 11C12 antibody or a labeled control IgG. Images of the mice were acquired on a Pearl imager 48- or 72-hours post antibody administration. Results from the studies are shown in FIG. 13 and demonstrate that 11C12, but not control antibodies, were able to specifically target tumor cells in the mice.

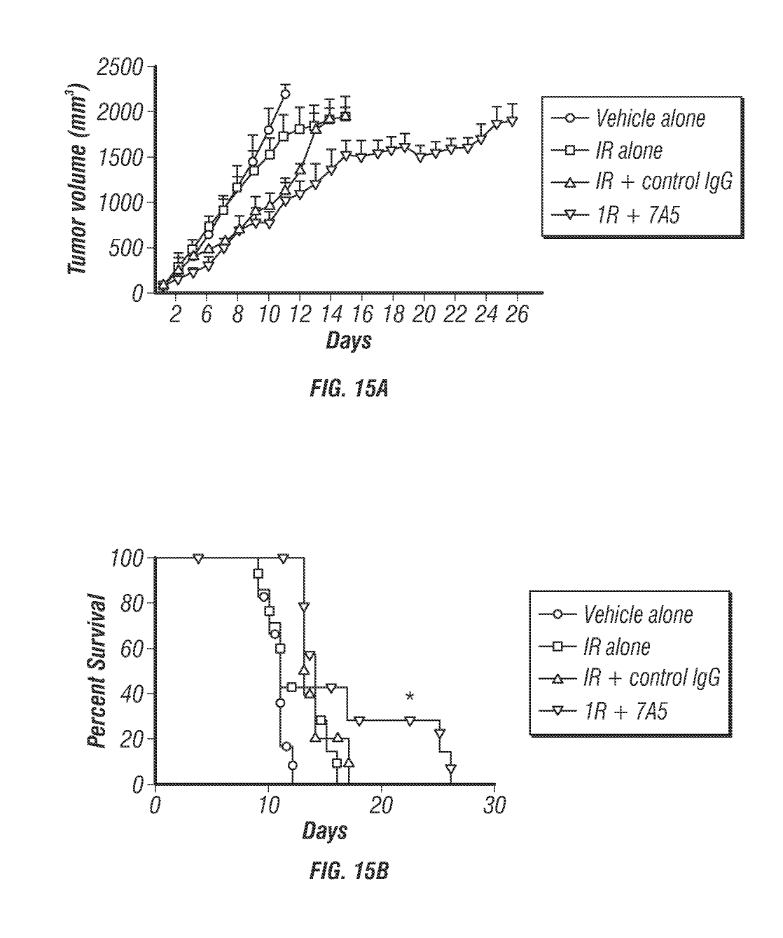

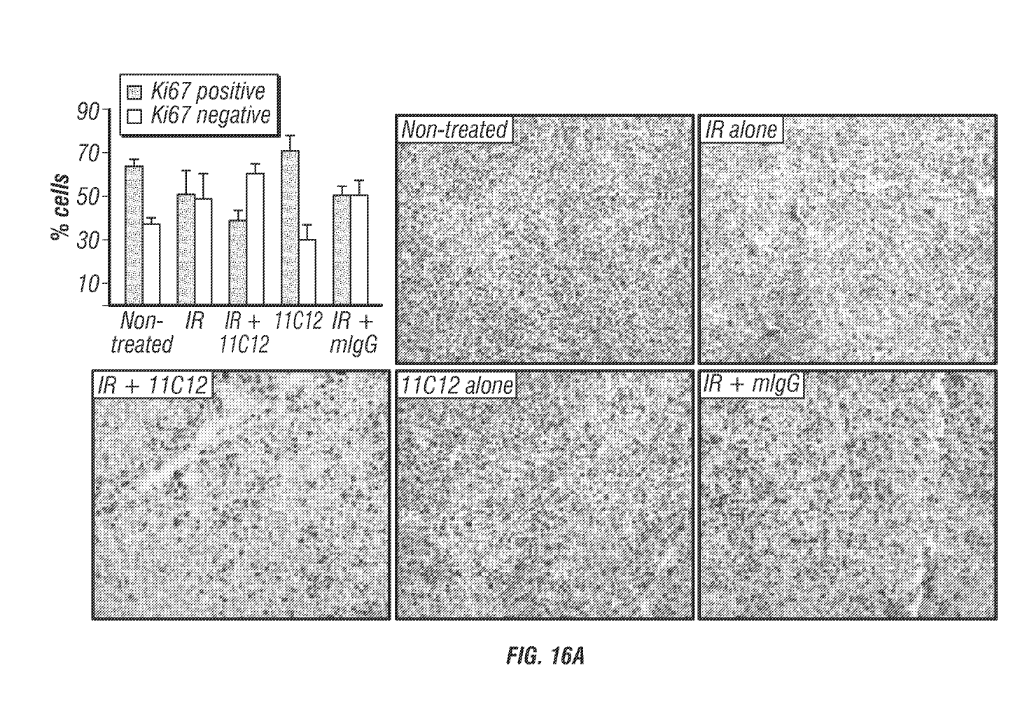

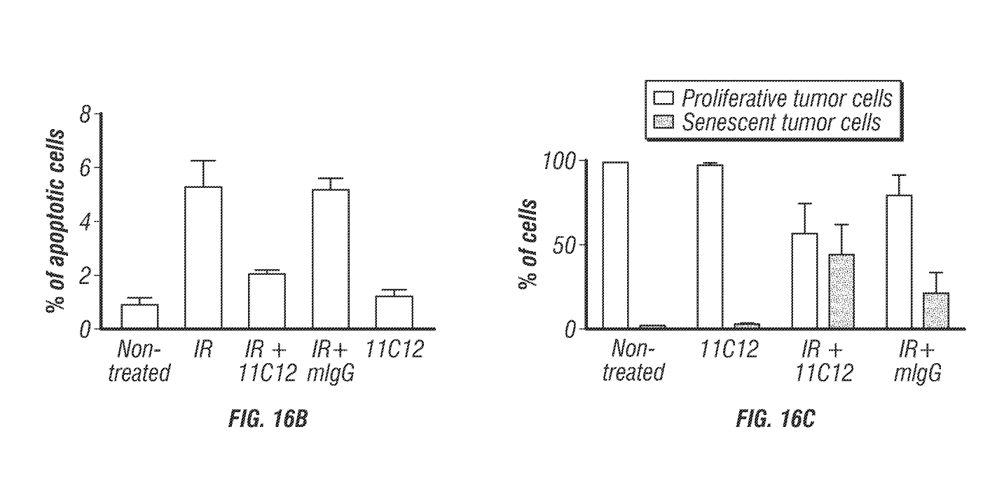

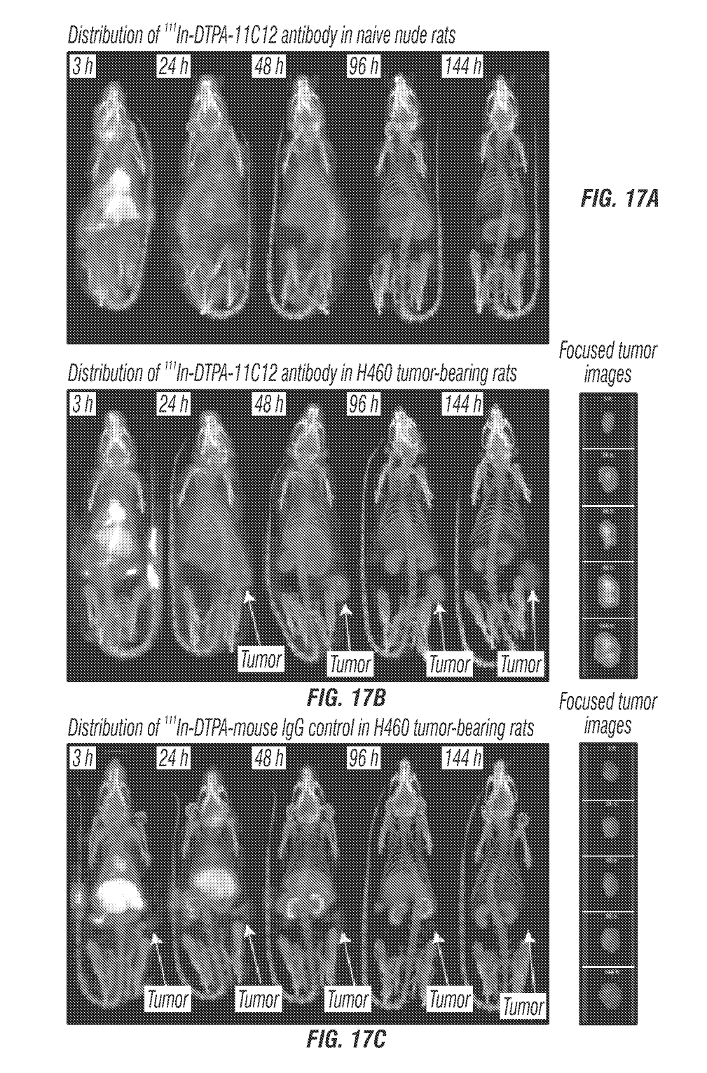

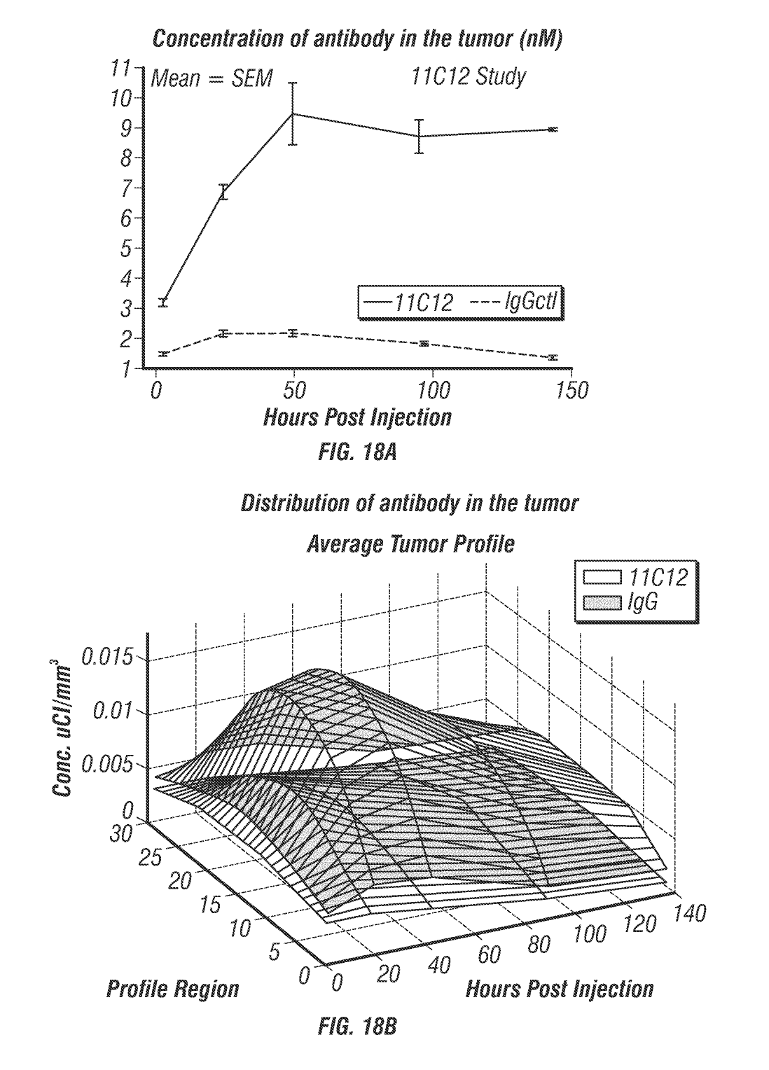

In vivo therapeutic effects of 11C12 and another EphA5 monoclonal antibody, 7A5 were also studied in a mouse model. For these experiments nude mice were inoculated with H460 cancer cells and tumor growth was assessed for a period of 30 or 26 days, respectively. Groups of at least 8 mice were treated with 5 mg/kg of monoclonal antibody (11C12 or 7A5) or control antibody either alone or in conjunction with radiation. Irradiation was applied 6 hours after antibody administration and was repeated at a dose of 3 Gy/day for three consecutive days (for a total 9 Gy radiation dose). Daily tumor volumes in the animals and animal survival were plotted and are shown in FIG. 14 and FIG. 15. These results show a significant reduction in tumor growth in animal treated with 11C12 or 7A5 and radiation as compared to both untreated animal or animals treated with either agent alone.

### Example 7

### EphA5 Expression in Squamous Cell Carcinomas of the Cervix and Head and Neck

Immunohistochemical staining was performed with a commercial antibody against EphA5 on squamous cell cervical cancer and head and neck cancer tissue microarrays (TMA) (n=135 and 74, respectively). As can be seen in Tables 2 and 3 below, EphA5 was found to be widely expressed in both human cervical cancer (24%) as well as head and neck cancer (57%).

TABLE 2
EphA5 Staining in Squamous Cell Carcinoma of the Cervix

| | |
|---|---|
| EphA5 Positive | EphA5 Negative |

| | n | (%) | n | (%) |
|---|---|---|---|---|
| Overall | 33 | 24% | 102 | 76% |
| FIGO stage | | | | |
| IB | 24 | 28% | 62 | 72% |
| IIA | 3 | 20% | 12 | 80% |
| IIB | 7 | 20% | 28 | 80% |
| Nodal Status | | | | |
| Negative | 25 | 24% | 81 | 76% |
| Positive | 8 | 28% | 21 | 72% |

TABLE 3
EphA5Staining in Squamous Cell Carcinoma of the Head and Neck

| | |
|---|---|
| EphA5 Positive | EphA5 Negative |

| | n | (%) | n | (%) |
|---|---|---|---|---|
| Overall | 42 | 57% | 32 | 43% |
| Primary Site | | | | |
| Oropharynx | 21 | 49% | 22 | 51% |
| Larynx | 14 | 67% | 7 | 33% |
| Hypopharynx | 4 | 67% | 2 | 33% |
| Other | 3 | 75% | 1 | 25% |
| T stage | | | | |
| T0-2 | 25 | 53% | 22 | 47% |
| T3,4 | 17 | 63% | 10 | 37% |
| N Stage | | | | |
| 0 | 18 | 69% | 8 | 31% |
| 1 | 2 | 33% | 4 | 67% |
| 2 | 22 | 54% | 19 | 46% |
| 3 | 0 | 0% | 1 | 100% |
| Stage | | | | |
| I | 2 | 67% | 1 | 33% |
| II | 6 | 60% | 4 | 40% |
| III | 19 | 70% | 8 | 30% |
| IV | 15 | 44% | 19 | 56% |

### Example 8

### Materials and Methods

Animals—

Balb/C mice (The Jackson Laboratories) were housed in the animal facility of the University of Texas M. D. Anderson Cancer Center (MDACC) in Houston, Tex. All animal procedures were reviewed and approved by the Institutional Animal Care and Use Committee of the MDACC.

Reagents—

The following reagents were used: rabbit polyclonal anti-EphA5 antibody (Santa Cruz Biotechnology, clone L15), rabbit monoclonal anti-ATM IgG (Millipore, clone Y170), and mouse monoclonal anti-phospho-ATM IgG (Ser1981) (Millipore, clone 10H11.E12), mouse monoclonal anti-BrdU IgG (Millipore), rabbit polyclonal anti-pTyr-horseradish peroxidase (HRP)-conjugated (Santa Cruz Biotechnology), and goat polyclonal anti-GST antibody (GE Healthcare Life Sciences). Goat anti-rabbit HRP-conjugated, rabbit anti-goat HRP-conjugated, and rabbit anti-mouse HRP-conjugated IgGs were purchased from Jackson ImmunoResearch Laboratories. Alexa Fluor® 488 rabbit anti-mouse IgG was purchased from Invitrogen. Cy-3- and FITC-conjugated anti-mouse and rabbit IgG were purchased from Jackson ImmunoResearch Laboratories. Recombinant human EphA5 extracellular domain was purchased from R&D Systems. The complete EphA5 cytoplasmic domain (Cellsciences) and EphA5 kinase domain (Millipore) were also commercially obtained.

Cell Culture—

The 293GPG cells were cultured in high glucose Dulbecco's modified Eagle's medium (DMEM) containing 10% heat-inactivated fetal bovine serum (FBS) and 1 μg/ml tetracycline (Sigma), at 37° C. and 5% CO₂. NCI-H460, and NCI-H522 human lung cancer cell lines were purchased from the American Type Culture Collection (ATCC) and maintained in DMEM containing 10% FBS and antibiotics. After lentiviral infection, stably transfected cells were selected and maintained in media containing 10 μg/ml puromycin (Sigma). Normal HPF were obtained from PromoCell and cultivated in supplemented fibroblast growth medium (PromoCell). Hybridomas were kept in RPMI-1640 supplemented with 10% FBS and antibiotics.

Plasmid DNA Constructs—

The full-length cDNA of human EPHA5 was purchased from GeneCopoeia™ (# EX-Y3268-LV47, NCBI accession No. NM_{—}182472, incorporated herein by reference and provided as SEQ ID NO:13).

Virus Preparations—

The lentiviral vectors (pCCLsin.PTT.PGK.EGFP.Wpre, pMDLg/pRRE, pRSV-Rev, and pMD2.VSVG) were a gift from Dr. L. Naldini (San Raffaele Hospital and Research Center, Milan, Italy), and the recombinant lentiviruses were produced as described (Salameh et al., Blood 106, 3423 (2005).). Briefly, 293FT cells were transiently transfected by using Lipofectamine 2000 (Invitrogen) for 16 h, after which the lentiviruses were harvested 24 and 48 h later and passed through 0.22 μm pore cellulose acetate filters. Recombinant lentiviruses were concentrated by ultracentrifugation for 2 h at 50,000 g. Vector infectivity was evaluated by cell infection with a GFP vector and by puromycin drug selection.

Protein Extracts, Immunoblotting, and Immunoprecipitation—

Extraction of total protein was performed with NP-40 extraction buffer containing protease inhibitors [50 mM Tris-buffer (pH 8.0), 150 mM NaCl and 1% NP-40]. For immunoprecipitation assays, cells were lysed in NP-40 extraction buffer and, after overnight (ON) incubation with anti-EphA5 antibody-loaded magnetic beads (2 mg of total protein/1 μg of antibody), washed extensively in wash buffer [50 mM Tris-buffer (pH 8.0) 0.5 M NaCl, and 1% NP-40]. For protein detection by immunoblotting, proteins were resolved on a 4-12% NuPAGE® Novex Bris-Tris gels (Invitrogen), transferred to nitrocellulose membranes (BioRad), blocked with phosphate-buffered saline (PBS) containing 5% non-fat milk (BioRad), incubated with primary antibody, washed, incubated with HRP-conjugated secondary antibody, and developed with an Enhanced Chemiluminescence reagent (GE Healthcare Life Sciences).

Subcellular Fractionation—

For cytosolic and nuclear extracts, cells were washed with ice-cold PBS and pelleted at 1,200 rpm for 3 min. Cells were resuspended in ice-cold hypotonic lysis buffer containing protease and phosphatase inhibitors [10 mM HEPES (pH 7.9), 60 mM KCl, 1 mM EDTA, and 0.5% NP-40)], and centrifuged at 500 g for 5 min at 4° C. The supernate containing the cytoplasmic proteins was transferred to a new tube. Nuclei were washed in hypotonic lysis buffer without NP-40 and resuspended in nuclear extraction buffer [250 mM Tris-HCl (pH 7.8), 60 mM KCl, 1 mM EDTA, 1 mM DTT, and 0.5% NP-40)]; the nuclear membrane was disrupted by gentle sonication. The nuclear extract was centrifuged at 15,000 g for 30 min at 4° C., and the supernate containing nuclear proteins was transferred to a new tube (19, 20).

ELISA-Based Protein Interaction and Solution Binding Assay—

Immunocapture of ATM and pATM was performed with Reacti-bind protein-A plates (Pierce) coated with anti-pATM, anti-ATM, or IgG control antibodies as described (see, e.g., Pasqualini et al., Cancer Res., 60, 722 (2000)). ELISA with anti-IgG confirmed equal molar concentrations of IgG in each of the wells. After a blocking step with PBS containing 3% BSA, 30 μg protein from total cell extracts was added to the wells for ON incubation at 4° C. After gentle washes, tagged EphA5 constructs were added to each well; after ON incubation and several washes in PBS, the assay was developed with HRP-conjugated secondary antibodies against the tags. Solution binding assays were performed in binding buffer [20 mM HEPES (pH 6.8), 150 mM KOAc, 2 mM Mg(OAc)2, 2 mM DTT, 0.1% Tween 20]. For each experiment, EphA5 constructs were incubated with anti-pATM, anti-ATM antibodies, or control IgG-coupled magnetic beads in 500 μL of binding buffer ON at 4° C. At the end of the incubation period, beads were collected by centrifugation, and unbound proteins in the supernate were collected by removal of 28 μL from the meniscus (corresponding to the unbound fraction). After two washes with 500 μL of binding buffer, the beads were resuspended in 20 μL of buffer. All samples were processed by addition of sample buffer containing β-mercaptoethanol and by heating at 95° C. for 10 min. Proteins were resolved in NuPAGE® gels, transferred to a nitrocellulose membrane, and analyzed by immunoblotting.

Clonogenic Assay—

Cells were removed and plated sparsely on 6-well plates (Falcon) (250 cells/well) or 100×15 mm Petri dishes (BD Falcon) (5,000 cells/dish), and were exposed to the specified doses of IR. Ten days later, cells were fixed and stained in methanol containing 0.5% crystal violet to facilitate counting of colonies (≧50 cells). Clonogenic survival was calculated for each IR dose after correction for plating efficiency.

ATM Foci Formation—

Cells were plated in 16-well chambers (Lab-Tek®) at 10,000 cell/well. After ON incubation, cells were treated with 3 Gy of IR and fixed in methanol at different time points after irradiation. Cells were blocked for 2 h with 1% bovine serum albumin (BSA) in PBS, followed by ON incubation with primary antibodies diluted in 1% BSA in PBS. After three washes with PBS, cells incubated for 30 min with FITC-conjugated anti-rabbit and Cy3-conjugated anti-mouse antibodies, diluted in 1% BSA in PBS. Cells were washed three times with PBS and were mounted with mounting reagent containing DAPI (Vectashield—Vector Labs). Cells were viewed under a 60× oil immersion objective. Images were recorded, pseudo-colored, and merged. For quantification, ATM foci in the nuclei of at least 50 cells were counted under the microscope for each experimental condition.

Cell Cycle Analysis—

Cells were pulsed with BrdU (Sigma) for 1 h prior to removal with trypsin. Aliquots were subsequently neutralized, washed in PBS, and fixed in ice-cold 70% ethanol under constant vortexing. After storage on ice, cells were vortexed into 2 N HCl/0.5% Tween-20. After 30 min of incubation, cells were washed twice in PBS/HEPES (pH 7.4), to restore physiological pH, and incubated with PBS containing 1% BSA, 0.5% Tween-20, and FITC-conjugated anti-BrdU antibody (Millipore), for 1 h at room temperature (RT). After several washes, cells were incubated in 50 μM PI dissolved in PBS, in the presence of DNAase-free RNAase (2.5 μg/ml, Sigma) for 30 min, in the dark. Samples were analyzed immediately thereafter by FACS (Becton-Dickenson). For evaluation of DNA content, cells were treated, removed and fixed in 70% ice-cold ethanol during vortexing. Approximately 30 min before analysis, cells were incubated in a solution of PBS containing 1% BSA and 50 μM PI.

Monoclonal Antibody Production—

Balb/c mice were immunized three times at 14 days intervals with 12 mg of recombinant extracellular EphA5 dissolved in complete Freund's adjuvant per immunization. Sera collected from tail veins were tested by ELISA after the third immunization. Splenocytes of mice with high antibody titers were fused with mouse myeloma cells (P3X63Ag8.653, ATCC) at a 4:1 ratio with polyethylene glycol (MW=1500; Sigma). After fusion, cells were cultured in 96-well plates at 1×10⁵ cells/well in RPMI 1640 selection medium containing 20% FBS, 10% hybridoma supplements (Sigma), 2 mM L-glutamine, 100 U/ml penicillin G, 100 mg/ml streptomycin SO₄, 10 mM HEPES, and hypoxanthine-aminopterin-thymidine (Sigma). Selected hybridomas were subcloned four times by limiting dilution. Conditional media were harvested from each stable hybridoma culture. The Ig class of mAb was then determined with a mouse mAb isotyping kit (Santa Cruz).

High-Density Tissue Microarrays and Immunohistochemistry—

Human non-small cell lung cancer TMA specimens were from the Lung Cancer Specialized Program of Research Excellence (SPORE) Tissue Bank at the MDACC (see, Prudkin et al., Clin Cancer Res 14, 41 (2008)). Prior to the construction of the TMA, tumor tissue specimens of 255 lung cancers (152 ACC, 94 SCC, and 9 other NSCLC histologies) were histologically examined and classified according to the WHO system. Detailed clinical and pathologic information, including demographic data, smoking history, pathologic tumor-node-metastasis staging, overall survival, and time of recurrence was available in most cases. EphA5 immunostaining was performed on an autostainer (Dako Corporation). Hematoxylin was used for counterstaining Immunohistochemical expression of EphA5 was quantified by two expert lung cancer pathologists (I. I. W. and M. I. N.) according to a four-value intensity score (0 for negative, 1 for weak, 2 for moderate, and 3 for strong), and the percentage of tumor cells within each category was independently estimated (0-100%). A final score was obtained by the multiplying of both intensity and extension values (0×% negative tumor cells+1×% weakly-stained tumor cells+2×% moderately-stained tumor cells+3×% strongly-stained tumor cells). Thus, the score ranged from a minimum of zero to a maximum of 300.

Evaluation of EphA5 expression in a second cohort of patients that had previously been treated with radiotherapy was also performed. All patient information and materials were collected and handled in accordance with Institutional Review Board (IRB)-approved protocols and procedures. Patients with stage IIB-IIIB NSCLC treated with primary lobectomy or pneumonectomy followed by post-operative radiotherapy at MDACC from 1998-2005 were considered for inclusion. Patients receiving radiation doses less than 50 Gy, with positive surgical margins, or without surgical mediastinal lymph node evaluation, were excluded. After a cross-referencing for archived tissue materials, there were 24 patients available for further analysis. Individual patient medical records were reviewed for pertinent demographic and treatment outcome data. Locoregional relapse was defined as biopsy-proven recurrence within the radiation field. After immunohistochemistry for EphA5 was performed, the cohort was divided into high versus low EphA5 expression on the basis of scores at or above versus below the median score (130) for the group. Cumulative rates of locoregional relapse were plotted, and log-rank analysis to detect differences between the groups. Overall survival was estimated for the cohort as a function of EphA5 staining by Kaplan-Meier methodology, by using log-rank analysis to detect differences between the groups (SPSS version 17).

Statistical Analyses—

Survival probability as a function of time was computed by the Kaplan-Meier methodology. The log-rank test was applied to compare patient survival time between groups. EphA5 expression was compared between two groups by using the Wilcoxon rank sum test or among three groups by using the Kruskal-Wallis test. Association between EphA5 positivity (zero versus greater than zero) and other covariates tested either by the Fisher's exact test or chi-squared test. P<0.05 was considered to be statistically significant. Statistical analyses were done with S-Plus and SPSS software.

In Vivo Imaging of Tumor-Bearing Mice—

Labeling of mAb (11C12 or 7A5) and control IgG was performed with the IRDye 800CW Protein Labeling Kit (LI-COR). Labeled antibodies were administered intraperitoneally (3 μg/mouse), and near infrared fluorescence imaging of live animals were acquired on an Odyssey Infrared Imaging System (LI-COR).

In Vivo Tumor Irradiation—

Gamma irradiation was locally delivered to tumors from a 60Co source, at the rate of 1 Gy/min with a custom head and body shielding. Tumor-bearing mice received fractionated radiation therapy of 3 Gy a day for 3 consecutive days, 6 h after treatment with either mAb (11C12 or 7A5) or control IgG (5 mg/kg each). Control groups received vehicle alone, irradiation alone or mAb (11C12 or 7A5) alone. Tumor volumes were determined from digital caliper measurements and reported as mean tumor volumes ±SD.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims. The entire contents of any reference that is referred to herein are hereby incorporated by reference.
PCT Publication No. 99/26299
PCT Publication No. WO2006/056464
European Patent 0 216 846
European Patent 0 256 055
European Patent 0 323 997
European Patent Application No. 89303964.4
U.S. Patent Publn. 2002/0168707
U.S. Patent Publn. 2003/0051263
U.S. Patent Publn. 2003/0055020
U.S. Patent Publn. 2003/0159161
U.S. Patent Publn. 2004/0019001
U.S. Patent Publn. 2004/0064842
U.S. Patent Appln. 2004/0161748
U.S. Patent Publn. 2004/0265839
U.S. Patent Publn. 2005/0106660
U.S. Patent Publn. 2006/0058510
U.S. Patent Publn. 2006/0088908
U.S. Patent Appln. 2007/0299246
U.S. Patent Appln. 2008/0293591
U.S. Patent Publn. 2010/0285564
U.S. Patent Publn. 2010/0317547
U.S. Patent Appln. 2011/0183863
U.S. Pat. No. 6,048,616
U.S. Pat. No. 5,990,479
U.S. Pat. No. 5,690,807
U.S. Pat. No. 5,505,928
U.S. Pat. No. 5,262,357
U.S. Pat. No. 3,817,837
U.S. Pat. No. 3,850,752
U.S. Pat. No. 3,939,350
U.S. Pat. No. 3,996,345
U.S. Pat. No. 4,277,437
U.S. Pat. No. 4,275,149
U.S. Pat. No. 4,366,241
U.S. Pat. No. 3,817,837
U.S. Pat. No. 3,850,752
U.S. Pat. No. 3,939,350
U.S. Pat. No. 3,996,345
U.S. Pat. No. 4,277,437
U.S. Pat. No. 4,275,149
U.S. Pat. No. 4,366,241
U.S. Pat. No. 4,196,265
U.S. Pat. No. 4,472,509
U.S. Pat. No. 4,938,948
U.S. Pat. No. 4,472,509
U.S. Pat. No. 4,938,948
U.S. Pat. No. 5,021,236
U.S. Pat. No. 5,196,066
U.S. Pat. No. 5,279,721
U.S. Pat. No. 5,208,020
U.S. Pat. No. 5,827,690
U.S. Pat. No. 5,756,687
U.S. Pat. No. 5,750,172
U.S. Pat. No. 5,741,957
U.S. Pat. No. 6,333,410
U.S. Pat. No. 6,506,559
U.S. Pat. No. 6,573,099
U.S. Pat. No. 6,569,620
U.S. Pat. No. 6,716,580
U.S. Pat. No. 6,673,611
U.S. Pat. No. 6,091,001
U.S. Pat. No. 7,276,497
Abbondanzo et al., 1990.
Allred et al., 1990.
Altschul, et al., Methods in Enzymology.
Altschul, et al., “Gapped BLAST and PSI-BLAST: a new generation of protein database search programs”, Nucleic Acids Res. 25:3389-3402, 1997.
Antibodies: A Laboratory Manual, Cold Spring Harbor Laboratory, 1988.
Ausubel, 1996.
Ausubel et al., 1998.
Baxevanis, et al., Bioinformatics: A Practical Guide to the Analysis of Genes and Proteins,
Wiley, 1998.
Berberian et al., 1993
Brown et al., 1990.
Cleary et al., 1994.
Cumber et al. (1992) J Immunology 149B:120-126.
De Jager R et al., 1993.
Dholakia et al., 1989.
Doolittle M H and Ben-Zeev O, 1999.
Eaton and Piekern, Ann. Rev. Biochem., 64:837-863 (1995).
Gefter et al., (1977).
Goding, 1986, pp. 60 61.
Goding pp. 71 74, 1986).
Green et al., Chem. Biol., 2:683-695 (1995).
Gulbis B and Galand P, 1993.
J. Mol. Biol., 215(3): 403-410, 1990.
Kang et al., 1988.
Khatoon et al., 1989.
King et al., 1989.
Klobmann et al., Nature Biotechnol., 14:1112-1115 (1996).
Kohl et al., PNAS, 2003, vol. 100, No. 4, 1700-1705.
Kohler and Milstein (1975; 1976). (in #99-not sure)
Kohler et al., 1989.
Kreier et al., 1991.
Lenert et al., 1990.
Liu et al. Cell Mol Biol (Noisy-le-grand). 2003 March; 49(2):209-16.
Misener, et al., (eds.), Bioinformatics Methods and Protocols (Methods in Molecular Biology, Vol. 132), Humana Press, 1999
Nakamura et al., 1987.
Nolte et al., Nature Biotechnol., 14:1116-1119 (1996).
O'Shannessy et al., 1987.
Owens & Haley, 1987; Atherton et al., 1985.
Pack et al. (1992) Biochem 31:1579-1584.
Pagratis et al., Nature Biotechnol., 15:68-73 (1997).
Pasqualini et al., Cancer Res., 60, 722 (2000).
Pitulescu et al. Genes & Dev., 24:2480-2492, 2010.
Potter & Haley, 1983.
Prudkin et al., Clin Cancer Res 14, 41 (2008).
Reed et al., Cancer Res. 50:6565-6570 (1990).
Salameh et al., Blood 106, 3423 (2005).
Sambrook et al., 1989.
Sasso et al., 1989.
Shorki et al., 1991.
Silvermann et al., 1995.
Skerra A., J. Mol. Recogn., 2000, 13:167-187.
Skerra A., J. Biotechnol., 2001, 74(4):257-75.
Song et al., 2003.
Soutschek et al., 2004; Wadhwa et al., 2004.
Tam et al., Nucl. Acids Res., 22:977-986 (1994).
Wadhwa et al., 2004.
Wood et al., Science, 291: 1284-1289 (2001).
Yoo et al., J Immunol Methods. 2002 Mar. 1; 261(1-2):1-20.

1-172. (canceled)

173. An isolated antibody, or an antigen-binding fragment thereof, characterized in that it binds to human EphA5 protein and characterized in that:
(a) it decreases levels of phosphorylated EphA5 in the nucleus of a cell expressing EphA5 as compared to a control;
(b) it decreases binding of EphA5 protein to pATM protein in a cell expressing EphA5 as compared to a control;
(c) it competes with the 11C12 antibody for binding to EphA5;
(d) it decreases cell growth, proliferation or cell survival in a cell expressing EphA5 as compared to a control; and/or
(e) any combination of (a)-(d).

174. The isolated antibody of claim 173, wherein the antibody specifically binds to a polypeptide of SEQ ID NO:3 or SEQ ID NO:6.

175. The isolated antibody of claim 173, wherein the antibody comprises a VH or VL amino acid sequence at least 80%, 99%, 95%, 98% or 99% identical to SEQ ID NO: 11 or SEQ ID NO: 12, respectively.

176. The isolated antibody of claim 173, wherein the antibody comprises a VH and VL amino acid sequence of SEQ ID NO: 11 and SEQ ID NO: 12, respectively.

177. A pharmaceutical composition comprising, a therapeutically effective amount of an agent that reduces the expression of the EphA5, reduces EphA5 phosphorylation or reduces the level of EphA5 in the nucleus of EphA5 expressing cells, wherein the agent comprises the isolated antibody, or the antigen-binding fragment thereof, of claim 173.

178. A method of producing an isolated antibody, or an antigen-binding fragment thereof, characterized in that it competes with an EphA5 ligand for binding to EphA5 comprising:
providing a cell expressing the isolated antibody; and
culturing the cell under conditions permissive for expression of the antibody thereby producing the antibody.

179. The method of claim 178, wherein the antibody specifically binds to a polypeptide of SEQ ID NO:3 or SEQ ID NO:6.

180. The method of claim 178, wherein the antibody comprises a VH or VL amino acid sequence at least 80%, 99%, 95%, 98% or 99% identical to SEQ ID NO: 11 or SEQ ID NO: 12, respectively.

181. The method of claim 180, wherein the antibody comprises a VH and VL amino acid sequence of SEQ ID NO: 11 and SEQ ID NO: 12, respectively.

182. A method of targeting delivery of an agent to an EphA5 ligand or an EphA5 expressing cell comprising:
providing an isolated antibody, or antigen binding-fragment thereof, that binds to human EphA5 protein;
linking the isolated antibody, or antibody binding-fragment thereof, to at least one agent; and
administering the agent linked to the isolated antibody, or antibody binding-fragment thereof, to a patient, thereby targeting delivery of the agent to the EphA5 ligand or the EphA5 expressing cell of the patient.

183. The method of claim 182, wherein the antibody specifically binds to a polypeptide of SEQ ID NO:3 or SEQ ID NO:6.

184. The method of claim 182, wherein the antibody comprises a VH or VL amino acid sequence at least 80%, 99%, 95%, 98% or 99% identical to SEQ ID NO: 11 or SEQ ID NO: 12, respectively.

185. The method of claim 184, wherein the antibody comprises a VH and VL amino acid sequence of SEQ ID NO: 11 and SEQ ID NO: 12, respectively.

186. The method of claim 182, wherein the agent comprises a toxin, an anti-tumor agent, a therapeutic enzyme, a radiolabeled nucleotide, an antiviral agent, a chelating agent, a cytokine, a growth factor, an oligonucleotide or a polynucleotide.

187. The method of claim 186, wherein the agent is an oligonucletide which is an inhibitory nucleic acid.


SEQUENCE LISTING (19 sequences)

SEQ ID NO: 1 - DNA, 361 bp, Artificial Sequence
  Monoclonal antibody coding sequence
gaggttactctgaaagagtctgggggaggcttagtgcagcctggagggtcccggaaactctcctgtgcagcctctggattcactttcagtacctttggaatacactgggttcgtcaggctccagagaagggactggagtgggtcgcatacattagtggtgccagtactaccatctactatgcagacacagtgaagggccgattcaccatctccagagacaatcccaagaacaccctgttcctgcaaatgaccagtctgaggtctgaggacacggccatgtattactgtgcaagatacggtactagcttcccctatggtttggactactggggtcaaggaacctcagtcaccgtctcctcag

SEQ ID NO: 2 - DNA, 333 bp, Artificial Sequence
  Monoclonal antibody coding sequence
gatattgtgatgacccaatctccaggttctttggctgtgtctctagggcagagggccaccatctcctgcaaggccagccaaagtgttgattatgatggtgatagttatatgaactggtaccaacagaaaccaggacagccacccaaactcctcatctatggtgcatccaatctagaatctgggatccctgccaggtttagtggcagtgggtctgggacagacttcaccctcaacatccatcctgtggaggaggaggatgctgcaagctattactgtcagcaaagtaatgaggatccattcacgttcggctcggggacaaagttggaaataaaa

SEQ ID NO: 3 - protein, 164 aa, Homo sapiens
VCRPGFFKASPHIQSCGKCPPHSYTHEEASTSCVCEKDYFRRESDPPTMACTRPPSAPRNAISNVNETSVFLEWIPPADTGGRKDVSYYIACKKCNSHAGVCEECGGHVRYLPRQSGLKNTSVMMVDLLAHTNYTFEIEAVNGVSDLSPGARQYVSVNVTTNQA

SEQ ID NO: 4 - protein, 24 aa, Homo sapiens
VCRPGFFKASPHIQSCGKCPPHSY

SEQ ID NO: 5 - protein, 48 aa, Homo sapiens
VCRPGFFKASPHIQSCGKCPPHSYTHEEASTSCVCEKDYFRRESDPPT

SEQ ID NO: 6 - protein, 72 aa, Homo sapiens
VCRPGFFKASPHIQSCGKCPPHSYTHEEASTSCVCEKDYFRRESDPPTMACTRPPSAPRNAISNVNETSVFL

SEQ ID NO: 7 - protein, 95 aa, Homo sapiens
VCRPGFFKASPHIQSCGKCPPHSYTHEEASTSCVCEKDYFRRESDPPTMACTRPPSAPRNAISNVNETSVFLEWIPPADTGGRKDVSYYIACKKC

SEQ ID NO: 8 - protein, 120 aa, Homo sapiens
VCRPGFFKASPHIQSCGKCPPHSYTHEEASTSCVCEKDYFRRESDPPTMACTRPPSAPRNAISNVNETSVFLEWIPPADTGGRKDVSYYIACKKCNSHAGVCEECGGHVRYLPRQSGLKN

SEQ ID NO: 9 - protein, 144 aa, Homo sapiens
VCRPGFFKASPHIQSCGKCPPHSYTHEEASTSCVCEKDYFRRESDPPTMACTRPPSAPRNAISNVNETSVFLEWIPPADTGGRKDVSYYIACKKCNSHAGVCEECGGHVRYLPRQSGLKNTSVMMVDLLAHTNYTFEIEAVNGV

SEQ ID NO: 10 - protein, 164 aa, Homo sapiens; identical to SEQ ID NO: 3

SEQ ID NO: 11 - protein, 120 aa, Artificial Sequence
  Antibody VH coding sequence
EVTLKESGGGLVQPGGSRKLSCAASGFTFSTFGIHWVRQAPEKGLEWVAYISGASTTIYYADTVKGRFTISRDNPKNTLFLQMTSLRSEDTAMYYCARYGTSFPYGLDYWGQGTSVTVSS

SEQ ID NO: 12 - protein, 111 aa, Artificial Sequence
  Antibody VL coding sequence
DIVMTQSPGSLAVSLGQRATISCKASQSVDYDGDSYMNWYQQKPGQPPKLLIYGASNLESGIPARFSGSGSGTDFTLNIHPVEEEDAASYYCQQSNEDPFTFGSGTKLEIK

SEQ ID NO: 13 - DNA, 7792 bp, Homo sapiens
gaggagccagaagcaaacttcagctgtctcagccggatccgtggttcctacatttggaggagccgcgtgccagaaggcgtaggaccccaaggggggacaaggaggactcccgagtctcccttcgccgctctgcgaggccgaagcggtggactgagccgctcgggacagcggcaccggaggaggctcggagaagatgcggggctcggggccccggggtgcgggacgccggcggcccccaagcggcggcggcgacacccccatcaccccagcgtccctggccggctgctactctgcacctcgacgggctcccctctggacgtgccttctcctgtgcgccgcactccggaccctcctggccagccccagcaacgaagtgaatttattggattcacgcactgtcatgggggacctgggatggattgcttttccaaaaaatgggtgggaagagattggtgaagtggatgaaaattatgcccctatccacacataccaagtatgcaaagtgatggaacagaatcagaataactggcttttgaccagttggatctccaatgaaggtgcttccagaatcttcatagaactcaaatttaccctgcgggactgcaacagccttcctggaggactggggacctgtaaggaaacctttaatatgtattactttgagtcagatgatcagaatgggagaaacatcaaggaaaaccaatacatcaaaattgataccattgctgccgatgaaagctttacagaacttgatcttggtgaccgtgttatgaaactgaatacagaggtcagagatgtaggacctctaagcaaaaagggattttatcttgcttttcaagatgttggtgcttgcattgctctggtttctgtgcgtgtatactataaaaaatgcccttctgtggtacgacacttggctgtcttccctgacaccatcactggagctgattcttcccaattgctcgaagtgtcaggctcctgtgtcaaccattctgtgaccgatgaacctcccaaaatgcactgcagcgccgaaggggagtggctggtgcccatcgggaaatgcatgtgcaaggcaggatatgaagagaaaaatggcacctgtcaagtgtgcagacctgggttcttcaaagcctcacctcacatccagagctgcggcaaatgtccacctcacagttatacccatgaggaagcttcaacctcttgtgtctgtgaaaaggattatttcaggagagagtctgatccacccacaatggcatgcacaagacccccctctgctcctcggaatgccatctcaaatgttaatgaaactagtgtctttctggaatggattccgcctgctgacactggtggaaggaaagacgtgtcatattatattgcatgcaagaagtgcaactcccatgcaggtgtgtgtgaggagtgtggcggtcatgtcaggtaccttccccggcaaagcggcctgaaaaacacctctgtcatgatggtggatctactcgctcacacaaactatacctttgagattgaggcagtgaatggagtgtccgacttgagcccaggagcccggcagtatgtgtctgtaaatgtaaccacaaatcaagcagctccatctccagtcaccaatgtgaaaaaagggaaaattgcaaaaaacagcatctctttgtcttggcaagaaccagatcgtcccaatggaatcatcctagagtatgaaatcaagtattttgaaaaggaccaagagaccagctacacgattatcaaatctaaagagacaactattactgcagagggcttgaaaccagcttcagtttatgtcttccaaattcgagcacgtacagcagcaggctatggtgtcttcagtcgaagatttgagtttgaaaccaccccagtgtttgcagcatccagcgatcaaagccagattcctgtaattgctgtgtctgtgacagtgggagtcattttgttggcagtggttatcggcgtcctcctcagtggaaggcggtgtggctacagcaaagcaaaacaagatccagaagaggaaaagatgcattttcataatgggcacattaaactgccaggagtaagaacttacattgatccacatacctatgaggatcccaatcaagctgtccacgaatttgctaaggagatagaagcatcatgtatcaccattgagagagttattggagcaggtgaatttggtgaagtttgtagtggacgtttgaaactaccaggaaaaagagaattacctgtggctatcaaaacccttaaagtaggctatactgaaaagcaacgcagagatttcctaggtgaagcaagtatcatgggacagtttgatcatcctaacatcatccatttagaaggtgtggtgaccaaaagtaaaccagtgatgatcgtgacagagtatatggagaatggctctttagatacatttttgaagaaaaacgatgggcagttcactgtgattcagcttgttggcatgctgagaggtatctctgcaggaatgaagtacctttctgacatgggctatgtgcatagagatcttgctgccagaaacatcttaatcaacagtaaccttgtgtgcaaagtgtctgactttggactttcccgggtactggaagatgatcccgaggcagcctacaccacaaggggaggaaaaattccaatcagatggactgccccagaagcaatagctttccgaaagtttacttctgccagtgatgtctggagttatggaatagtaatgtgggaagttgtgtcttatggagagagaccctactgggagatgaccaatcaagatgtgattaaagcggtagaggaaggctatcgtctgccaagccccatggattgtcctgctgctctctatcagttaatgctggattgctggcagaaagagcgaaatagcaggcccaagtttgatgaaatagtcaacatgttggacaagctgatacgtaacccaagtagtctgaagacgctggttaatgcatcctgcagagtatctaatttattggcagaacatagcccactaggatctggggcctacagatcagtaggtgaatggctagaggcaatcaagatgggccggtatacagagattttcatggaaaatggatacagttcaatggacgctgtggctcaggtgaccttggaggatttgagacggcttggagtgactcttgtcggtcaccagaagaagatcatgaacagccttcaagaaatgaaggtgcagctggtaaacggaatggtgccattgtaacttcatgtaaatgtcgcttcttcaagtgaatgattctgcactttgtaaacagcactgagatttattttaacaaaaaaagggggaaaagggaaaacagtgatttctaaaccttagaaaacatttgcctcagccacagaatttgtaatcatggttttactgaagtatccagttcttagtccttagtcttcatttttcatgaagcaaacatatcttgcattaaaagggacatgaagttagacatcatcttaagttacaacaacagaatccttcccactacttctacaaaattttgtacatgaaatatataattatatagcacttttatagactgaattaaggcaacccctttcaaaacttccagggatctacttgaaaggaaatgttttatagccatttgtgagctaacaaaagctacagtttactgaagtttacttcaagtcttaattgtctacaaaagtgtattgaagagcaatatgattagattatttcttaatagatatcttcttttgtaattttaaaatgctgttacacagcgttaagttatagaaactagtgtgtaaacatgttgcttgatcaagaaaaagtacaatacagggtgtatatttattttttgtgttataaagtttacttttagttgctcttctagagattattaggtaataaatgtgtatatactgtataatttgcaatatacccaggaactgatttaagatggaattgtgtgtgtgtttgcttgcacatgtgtgtgttaccattctgcttgcatttctaatagtgtttcaattttagcaacatataggagcaagtgttccagaatgtaatatgaataggagaaatagggaagcagtaaaacaaaatttaacacaagcttgtgtctcttttcctctcatgtgtccaaaagctaatctctttattcactaaaaggaaatgtgtataagactaaatcccctttggctttttaaaaacattttgtgatatcagtgacaatgcagttcttctagccattaatcttgtacccctgtaagaaatttcacctttctgagtcctgaaaagtatcttgtcaagcaaagttgacaccgaagggcacattttcagcaggatgtagaaggatttaactgtgcaggcttctgttaatgttgttaaatccaggcacatagcacgaagcatcacccttaagtgttaatccgttgtaaccattcccattttgactcagttctagaaattttgactctaaggcagcaatggaattatgacaaatatatatatatacacatacacacacacaaatatatatatatgcttatttcccttcagaattttatttcaataattgataagttttatttttaatggatgtttattcatttgttaatttcagtttgtattcagttacatggctttgagtttattttattgaaccaatccaaggtattatgtaattaggcttattaaggaatataacatatcttctatgtatgctctatataccacacatgatgaagacttatatttgtgatacaaattatgcagtgtattagaaattgtccaaaactgagctcctgagataaccaacatttttgtattattacactttataaaaattatggaatgtcatgtgattagtcaaaatcagagcatttaaataaaatacagcctactcatgattatcaatgttaatagtacagtagatcagagaagcctggattaatgaaatttatcagaaatatagccacgttatattaatggtattcttttttccaagcacttgttttatttgaatattagcacatataagaataaacttttgggaaataactacttcctattctgcccaccctttacttaagcagtaaagtacctcttttctaaagaaccaggaaacatcaaaataaccacaacaaaaatctaaacagttttcaaataatacattcagttctcataataataagggctatataccatgggtccacacaaactgtgctgcaaacatataatctattagtttagtaaaatttccagagatgtgaatttaatattttcttgataaaatcataaaaaagcatcaccattattaaagatgcatttgttcatttcaataaacagaaattaatgaaacaaattacttttgtacaaaataaatgatgattatgtggcactttattgaattttatagaaatattccaatctaagtgataaagtaacaactgagaaacttcagaatgaaagccttattgattcaaaaggaaaattaccatatgctttagggctgatgaacttgccacaattgcttagacataacaaagatattttctacattgttttccaactttatatgctgaaagataattaggaggtccgggtggagtgtatctaaatgactagaactttaagttgcaatatagattttctctttttaacaaaggaagaatataaattaatttggatcaaatttatttgccttctttgcaatcttggtgatcattttggaaagtaaattgaaaggaaagttaaatagccacatagttttcttttgcatctcaatttggttgagaatttctaaggaaggttaatatgactttagaatgaataaagagactgtcaaacaacaagtactctccctaaaaaagaggaaagaatgccttcctaaaatatttgtttcctattgtatatgtcaccaattggaaaatgccaatctcatctgcaagtgtgagaaatggaacaaggagaggagccaactctagtaaaatgtagtgaggccataatccaaggcaaagtttaacaaatcttgattgtgtaatttcttattctgttgattcattttgtcacccaccagactaccaaaaataaagcacagacatgacaattttagtatatacaacagatttggcaaataatacataatcttattgtaattgccagtagcaatatcttaaggggcatgagtctttcacagtattggactttgaaaaattggtacagggagtaattttcaacagactgatacttgaagtcactcaattactactttgacctatcataatttgtttctaatcttgtcatttgctcatatatactattaatataaccagttgctcttttcataatgtactatttctatgtaaagatgtaaagattttgattttcctatgttttaaaattgtaatgtgcatgatcttttatttcttgtgtttcaattaccaagtcagtattctaaggttgtaagattactgtaaaaattatattccaatattttatctaaacttaagtgtggccagcattattggtttcagagttgcctaaacaacacagaatatccttaattcagtttgtaaaagagttttgaccagtgtaaaaaagtgaagacacagtttcatgtttcaagtttaaaatggaaagttaatatcatttgagcacttatgtgttttcatgtcttctacttaatctgtatgtgaaagtaaaatatttttaacaccattatttatgccatgtaaagtgggttctcagaagcacaagcacagatatttaccttctgaagacatttttggctataaaagtgcattggatggcaaacactatttgagtcaggtgttagaaatttattagatgtattatattacatagtagaataaggtccctttctcattttgtttttcctaaaaataagaaaaaaaagaagtgtgatagtaaactgtctttgctaatgtcctgccagatgtttaacttcaaattaagcaaaaagtaggtacaatatgatgatgatcctgatgatgatgatgatgatgatgatgaagaataaatggaatcaaaatgctagctttatttgacattaataagtaaaataagtcatcattttttcaactctgtagcacagctgtttacattgaataattttctctattgtgctgttaattatatagtaatgtatcaaaatagaaataggaactttcttttcatttcttattcatttatgaagaaatcttttcacacataaatgattgaatgttcttgttcagaatgaccacataatcattgcttagaagaagataccaattcttttaaaagaaaaaaagtctgtttataataatcagaatcaaatgttgtttgtttcttctaaacgttaatggagaaaattgaaggtggtaaaatgtcatgtttattcaggctgggaactgtattcacagtagaagtttcagtggtcaacatatctatgactctttaggctgctgtagttttacagtcaattatttaaaagtgagtagttacatttataagagcctgagaatacttagactcagtcatttgttagtatttttaccaaaatctcttagtttcagacatgtcagaagcagctatatagcatatcttattctatgatatacatcaggctatctcaagttcctgtctcacagttaattcaaagaaggattaggatttctgtattttttctcatttgaatctttatgtgcatttggtttgtgtacatgctttttgtagtgtaagatatgaaattttatattttttcagaaaataaaaaccctttgaatacagtta

SEQ ID NO: 14 - protein, 5 aa, Artificial Sequence
  VH CDR 1
TFGIH

SEQ ID NO: 15 - protein, 17 aa, Artificial Sequence
  VH CDR 2
YISGASTTIYYADTVKG

SEQ ID NO: 16 - protein, 11 aa, Artificial Sequence
  VH CDR 3
YGTSFPYGLDY

SEQ ID NO: 17 - protein, 15 aa, Artificial Sequence
  VL CDR 1
KASQSVDYDGDSYMN

SEQ ID NO: 18 - protein, 7 aa, Artificial Sequence
  VL CDR 2
GASNLES

SEQ ID NO: 19 - protein, 7 aa, Artificial Sequence
  VL CDR 3
QQSNEDP